SEQUENCE LISTING (14 sequences)

SEQ ID NO: 1 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaatgggaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 2 - protein, 1049 aa, synthetic construct
MTIKEMPQPKTFGELKNLPLLNTDKPVQALMKIADELGEIFKWEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 3 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgaggccattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 4 - protein, 1049 aa, synthetic construct
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEAIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 5 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggaattttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 6 - protein, 1049 aa, synthetic construct
MTIKEMPQPKTFGELKNLPLLNTDKPVQALMKIADELGEIFKFEAPGRVTRYLSSQRLIKEACDESRFDKNLSQALKFVRDFAGDGNFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 7 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaattttggcgtgattttgcaggagacgggaattttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 8 - protein, 1049 aa, synthetic construct
MTIKEMPQPKTFGELKNLPLLNTDKPVQALMKIADELGEIFKFEAPGRVTRYLSSQRLIKEACDESRFDKNLSQALKFWRDFAGDGNFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 9 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgaataaatttgtacgtgattttgcaggagacgggaattttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 10 - protein, 1049 aa, synthetic construct
MTIKEMPQPKTFGELKNLPLLNTDKPVQALMKIADELGEIFKFEAPGRVTRYLSSQRLIKEACDESRFDKNLSQANKFVRDFAGDGNFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 11 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaagtcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 12 - protein, 1049 aa, synthetic construct
MTIKEMPQPKTFGELKNLPLLNTDKPVQALMKIADELGEIFKFEAPGRVSRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 13 - DNA, 3150 bp, Priestia megaterium
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 14 - protein, 1049 aa, Priestia megaterium [sequence omitted]

(19) United States
(12) Patent Application Publication
(10) Pub. No.: US 2024/0352431 A1
(43) Pub. Date: Oct. 24, 2024

(54) Variant Cytochrome P450 Monooxygenases and Uses Thereof

(71) Applicant: BluumBio Inc., Berkeley, CA (US)

(72) Inventors: Katherine French, Albany, CA (US); Azion White, Oakland, CA (US); Andrew Hendrickson, Lafayette, CA (US); Jordan James Baker, Oakland, CA (US)

(73) Assignee: BluumBio Inc., Berkeley, CA (US)

(21) Appl. No.: 18/634,603

(22) Filed: Apr. 12, 2024

Related U.S. Application Data
(60) Provisional application No. 63/497,297, filed on Apr. 20, 2023.

(51) Int. Cl. C12N 9/02; B09C 1/10; C02F 3/34

(52) U.S. Cl. CPC C12N 9/0071; B09C 1/10; C12Y 114/14001; C02F 3/342; C02F 3/344

(57) ABSTRACT

The present disclosure provides variant cytochrome P450 monooxygenase for the remediation of crude oil, PFAs, PCBs, and other organo-halides, polynucleotides encoding the variant cytochrome P450 monooxygenase, host cells expressing the variant cytochrome P450 monooxygenase, and methods of using the variant cytochrome P450 monooxygenase. Host cells can comprise the variant cytochrome P450 monooxygenase, almA (flavin binding monooxygenase), and xylE (catechol dioxygenase) for remediation of crude oil, PFAs, PCBs, and other organo-halides.

### REFERENCE TO SEQUENCE LISTING, TABLE OR COMPUTER PROGRAM

The official copy of the Sequence Listing is submitted concurrently with the specification as an xml file, made with WIPO Sequence Version 2.1.0, via EFS-Web, with a file name of “BBI003.xml”, a creation date of Mar. 31, 2024, and a size of 42 kilobytes. The Sequence Listing filed via EFS-Web is part of the specification and is incorporated in its entirety by reference herein.

### BACKGROUND

The global population continues to rise at an astonishing rate, with estimates suggesting it will be in excess of 9 billion in 2050. The intensive agricultural and industrial systems needed to support such a large number of people will inevitably cause an accumulation of soil, water and air pollution. Estimates have attributed pollution to 62 million deaths each year, 40% of the global total, while the World Health Organization (WHO) have reported that around 7 million people are killed each year from the air they breathe. Water systems fare little better, with an estimated 70% of industrial waste dumped into surrounding water courses. The world generates 1.3 billion tons of rubbish every year, the majority of which is stored in landfill sites or dumped into the oceans.

Oil spills in recent decades have left a long-term mark on the environment, ecosystem functioning, and human health. In the Niger Delta alone, the roughly 12,000 spills since the 1970s have left wells contaminated with benzene levels 1,000× greater than the safe limit established by the World Health organization and have irreparably damaged native mangrove ecosystems. Continued economic reliance on crude oil and legislation supporting the oil industry mean that the threat of spills is unlikely to go away in the near future.

At present, there are few solutions to cleaning up oil spills. Current approaches to removing crude oil from the environment include chemical oxidation, soil removal, soil capping, incineration, and oil skimming (in marine contexts). While potentially a ‘quick fix,’ none of these solutions are ideal. Soil removal can be costly and simply moves toxic waste from one site to another. Chemical oxidants can alter soil microbial community composition and pollute groundwater. Incineration can increase the level of pollutants and carbon dioxide in the air and adversely affect human health. Practices such as skimming only remove the surface fraction of the oil while the water-soluble portion cannot be recovered, negatively affecting marine ecosystems.

Cleaning up environmental contamination from human activities is one of the greatest unmet challenges of the twenty-first century.

### SUMMARY

Disclosed herein are variant cytochrome P450 monooxygenases which increase the reaction rate of cytochrome P450 monooxygenase with crude oil components and/or crude oil degradation intermediates. Variant cytochrome P450 monooxygenase disclosed herein have one or more amino acid substitutions made at amino acid positions L20, P25, V26, L29, F42, R47, V48, T49, Y51, A74, L75, V78, A82, L86, F87, W96, H138, V178, F181, A184, L188, H236, M237, E252, R255, F261, A264, T260, I263, T268, A290, A295, A328, P329, A330, L353, M354, P382, S383, A384, I385, P386, Q387, F393, I401, F405, L437, or T438. Variant cytochrome P450 monooxygenases disclosed herein can have one or more of the following amino acid substitutions, F42W, H138A, L86N, V78W, L75N, T49S, T49A, F42T, F42V, F42C, F42G, F42I, F42R, and F42D. Variant cytochrome P450 monooxygenases disclosed herein can have the amino acid sequence of SEQ ID NO: 2, 4, 6, 8, 10, or 12. Variant cytochrome P450 monooxygenases disclosed herein can have the nucleic acid sequence of SEQ ID NO: 1, 3, 5, 7, 9, or 11.

Variant cytochrome P450 monooxygenases disclosed herein can have at least one of the following amino acid changes F42W, H138A, L86N, V78W, L75N, T49S, T49A, F42T, F42V, F42C, F42G, F42I, F42R, and F42D, and the variant can have 70%, 80%, 90%, 95% or 99% sequence identity with SEQ ID NO: 14. Variant cytochrome P450 monooxygenases disclosed herein can have at least one of the following amino acid changes F42W, H138A, L86N, V78W, L75N, T49S, T49A, F42T, F42V, F42C, F42G, F42I, F42R, and F42D, and the variant can be encoded on a nucleic acid having 70%, 80%, 90%, 95% or 99% sequence identity with SEQ ID NO: 13.

Also disclosed herein are nucleic acids encoding the variant cytochrome P450 monooxygenases. The nucleic acids encode the variant cytochrome P450 monooxygenase polypeptides described above. The nucleic acids can encode variant cytochrome P450 monooxygenases with one or more amino acid substitutions made at amino acid positions L20, P25, V26, L29, F42, R47, V48, T49, Y51, A74, L75, V78, A82, L86, F87, W96, H138, V178, F181, A184, L188, H236, M237, E252, R255, F261, A264, T260, I263, T268, A290, A295, A328, P329, A330, L353, M354, P382, S383, A384, I385, P386, Q387, F393, I401, F405, L437, or T438. The nucleic acids can encode variant cytochrome P450 monooxygenases with one or more of the following amino acid substitutions, F42W, H138A, L86N, V78W, L75N, T49S, T49A, F42T, F42V, F42C, F42G, F42I, F42R, and F42D. The nucleic acids encoding variant cytochrome P450 monooxygenases disclosed herein can have the nucleic acid sequence of SEQ ID NO: 1, 3, 5, 7, 9, or 11.

Nucleic acids encoding variant cytochrome P450 monooxygenases include those that hybridize under stringent hybridization conditions to the nucleic acids of SEQ ID NO: 1, 3, 5, 7, 9, or 11. Nucleic acids encoding variant cytochrome P450 monooxygenases include those that encode a variant with at least one of the following amino acid changes F42W, H138A, L86N, V78W, L75N, T49S, T49A, F42T, F42V, F42C, F42G, F42I, F42R, and F42D, and which nucleic acid hybridizes under stringent hybridization conditions to the nucleic acids of SEQ ID NO: 1, 3, 5, 7, 9, or 11.

Nucleic acids described herein include constructs and expression constructs that can be transferred from a donor host cell to a recipient host cell. Transfer of the constructs can be done by conjugation, transfection or transformation. Conjugation systems used herein can be promiscuous systems able to transfer nucleic acids from a donor cell to many different types of recipient cells. For example, IncA/C conjugative plasmids have a broad host range which include members of Beta-, Gamma-, and Delta-proteobacteria classes. IncP and IncPromA conjugative plasmids can transfer to a broad range of soil bacteria including recipients from eleven (11) different bacterial phyla.

Host cells for the variant cytochrome P450 monooxygenases include, for example, soil bacteria, petroleum degrading bacteria, petroleum degrading plants, enteric bacteria, etc. Examples of petroleum degrading bacteria include, for example, Achromobacter (e.g., Achromobacter xylosoxidans DN002), Acinetobacter (e.g., sp. RAG-1), Aeromonas (e.g., A. hydrophila), Agmenellum (e.g., quadruplicatum), Alcanivorax, Alcaligenes (e.g., A. xylosoxidans), Alkanindiges, Alteromonas, Arthrobacter, Bacillus (e.g., B. Megaterium, B. subtilis, B. licheniformis), Burkholderia, Cycloclasticus, Dietzia (e.g., Dietzia sp. DQ12-45-1b), Enterobacter, Gordonia sp., Kocuria, Marinobacter, Mycobacterium, Ochrobactrum, Oleispira, Pandoraea, Pseudomonas (e.g., P. aeruginosa, P. luorescens, P. putida), Rhodococcus (e.g., R. equi), Staphylococcus, Stenotrophomonas (e.g., S. maltophilia), Streptobacillus, Streptococcus, Thallassolituus, and Xanthomonas sp.

In an aspect, the variant cytochrome P450 monooxygenase can increase the growth rate of Bacillus megaterium when it utilizes crude oil or dodecane as a carbon source, as compared to Bacillus megaterium transformed with a nucleic acid encoding wild-type cytochrome P450 monooxygenase. The variant cytochrome P450 monooxygenase also can have increased activity for degradation of crude oil, dodecane, and/or benzopyrene as compared to wild-type cytochrome P450 monooxygenase.

The variant cytochrome P450 monooxygenase described herein can be used for the remediation of crude oil contamination in the environment. For example, nucleic acids encoding the variant cytochrome P450 monooxygenase, almA (flavin binding monooxygenase), and xylE (catechol dioxygenase) can be engineered into a host cell to increase the host cell's ability to bioremediate crude oil, dodecane, and/or benzopyrene in the environment (e.g., the soil). Constructs expressing the variant cytochrome P450 monooxygenase, almA (flavin binding monooxygenase), and xylE (catechol dioxygenase) can be engineered into bacterial host cells or plant host cells. The bacterial and/or plant host cells can then utilize crude oil or crude oil components as a carbon source. When bacterial host cells are used, the construct can be designed so it is transferred by conjugation to other bacterial cells present in the contaminated environment (e.g., soil bacteria). This imparts to the soil bacteria genes/enzymes that improve the ability of those soil bacteria to utilize crude oil or crude oil components as a carbon source.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As used in this specification and the appended claims, the singular forms “a”, “an” and “the” include plural referents unless the context clearly indicates otherwise. Thus, for example, reference to “a polypeptide” includes more than one polypeptide.

The section headings used herein are for organizational purposes only and not to be construed as limiting the subject matter described.

### Definitions

As used herein, the terms “protein”, “polypeptide,” and “peptide” are used interchangeably and are defined to mean a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids. In some embodiments of the descriptions of polypeptides, the standard single or three letter abbreviations are used for the genetically encoded amino acids (see, e.g., IUPAC-IUB Joint Commission on Biochemical Nomenclature, “Nomenclature and Symbolism for Amino Acids and Peptides,” Eur. J. Biochem. 138:9-37, 1984).

As used herein, the terms “polynucleotide” or “nucleic acid’ are used interchangeably and are defined to mean two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. Preferably, such modified or synthetic nucleobases will be encoding nucleobases.

As used herein, the term “coding sequence” is defined to mean a portion of a nucleic acid (e.g., a gene) that encodes an amino acid sequence of a protein.

As used herein, the terms “wild-type” is defined to mean the form found predominantly in nature. For example, a wild-type polypeptide or polynucleotide sequence is a sequence predominantly present in an organism that can be isolated from a source in nature and which has not been intentionally modified by human manipulation.

As used herein, the terms “recombinant” or “engineered” or “non-naturally occurring” are used interchangeably and are defined to mean modified polypeptides or nucleic acids which polypeptides or nucleic acids are modified in a manner that would not otherwise exist in nature, or is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, the terms “percentage of sequence identity” and “percentage homology” are used interchangeably and are defined to mean comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25 (17): 3389-3402, 1977; respectively. Software for performing BLAST analyses is publicly available through the National Center for Biotechnology Information website. BLAST for nucleotide sequences can use the BLASTN program with default parameters, e.g., a wordlength (W) of 11, an expectation (E) of 10, M=5, N=−4, and a comparison of both strands. BLAST for amino acid sequences can use the BLASTP program with default parameters, e.g., a wordlength (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison WI), using default parameters provided.

As used herein, the term “reference sequence” is defined to mean a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In an aspect, a “reference sequence” can be based on a primary amino acid sequence, where the reference sequence is a sequence that can have one or more changes to the primary sequence.

As used herein, the term “substantial identity” refers to a polynucleotide or polypeptide sequence that has at least 80 percent sequence identity, at least 85 percent identity and 89 to 95 percent sequence identity, more usually at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity). Preferably, residue positions which are not identical differ by conservative amino acid substitutions.

As used herein, the terms “corresponding to”, “reference to” or “relative to” are used interchangeably when used in the context of the numbering of a given amino acid or polynucleotide sequence and are defined in this context to mean the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of a variant cytochrome P450 monooxygenase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. As such, the term “corresponding to”, “reference to” or “relative to” also refers to a residue that is analogous, homologous, or equivalent to an enumerated residue in a reference polypeptide. In addition, in some embodiments, crystal structure coordinates of a reference sequence may be used as an aid in determining a homologous polypeptide residue's three dimensional structure and location of equivalent residues.

As used herein, the terms “consensus sequence” and “canonical sequence” are defined to mean an archetypical amino acid sequence against which all variants of a particular protein or sequence of interest are compared. The terms also refer to a sequence that sets forth the nucleotides that are most often present in a DNA sequence of interest. For each position of a gene, the consensus sequence gives the amino acid that is most abundant in that position in a multiple sequence alignment (MSA).

As used herein, the terms “optimal alignment” or “optimally aligned” are defined to mean the alignment of two (or more) sequences giving the highest percent identity score. For example, optimal alignment of two polypeptide sequences can be achieved by aligning the sequences such that the maximum number of identical amino acid residues in each sequence are aligned together or by using software programs or procedures described herein or known in the art. Optimal alignment of two nucleic acid sequences can be achieved by aligning the sequences such that the maximum number of identical nucleotide residues in each sequence are aligned together. Two sequences (e.g., polypeptide sequences) may be deemed “optimally aligned” when they are aligned using defined parameters, such as a defined amino acid substitution matrix, gap existence penalty (also termed gap open penalty), and gap extension penalty, so as to achieve the highest similarity score possible for that pair of sequences. Optimal alignment can be done manually or by using software programs or procedures described herein or known in the art. e.g., the BLASTP program for amino acid sequences and the BLASTN program for nucleic acid sequences.

As used herein, the terms “amino acid substitution” or “amino acid difference” are defined to mean a change in the amino acid residue at a position of a polypeptide sequence relative to the amino acid residue at a corresponding position in a reference sequence which is the primary translation product starting at the methionine initiation codon. The positions of amino acid differences generally are referred to herein as “Xn,” where n refers to the corresponding position in the reference sequence upon which the residue difference is based. For example, a “residue difference at position X as compared to the primary translation product starting at the methionine initiation codon” refers to a change of the amino acid residue at the polypeptide position corresponding to position X of a wild-type protein. Thus, if the reference polypeptide of the primary translation starting at the methionine initiation codon product for a wild type gene has a valine at position X, then an “amino acid substitution” or “residue difference at position X as compared to reference sequence” refers to an amino acid substitution of any residue other than valine at the position of the polypeptide corresponding to position X of the reference sequence. In most instances herein, the specific amino acid substitution or amino acid residue difference at a position is indicated as “XnY” where “Xn” specifies the corresponding position as described above, and “Y” is the single letter identifier of the amino acid found in the engineered polypeptide (i.e., the different residue than in the reference polypeptide). In an aspect, where more than one amino acid can appear at a specified residue position, the alternative amino acids can be listed in the form XnY/Z, where Y and Z represent alternate amino acid residues. In some instances, the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present disclosure can include one or more amino acid residue differences relative to a reference sequence, which is indicated by a list of the specified positions where changes are made relative to the reference sequence.

As used herein, the terms “conservative amino acid substitution” or “conservative amino acid difference” are defined to mean a change in the amino acid at a residue position to a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, and isoleucine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively. Exemplary conservative substitutions are provided in Table 1 below.

TABLE 1

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

As used herein, the terms “non-conservative substitution” or “non-conservative amino acid difference” are defined to mean a change in the amino acid at a residue position to a different residue with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, the term “deletion” is defined to mean a modification of a polypeptide by removal of one or more amino acids from the reference polypeptide or modification of a nucleic acid by removal of one or more nucleotides from the reference nucleic acid. For example, deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of a variant cytochrome P450 monooxygenase. Deletions can be directed to the internal portions and/or terminal portions of the polypeptide. In various embodiments, the deletion can comprise a continuous segment or can be discontinuous.

As used herein, the term “insertion” is defined to mean a modification to a polypeptide by addition of one or more amino acids from the reference polypeptide, or modification of a nucleic acid by addition of one or more nucleic acids. The improved variant cytochrome P450 monooxygenase comprise insertions of one or more amino acids to a cytochrome P450 monooxygenase polypeptide, which can include variant cytochrome P450 monooxygenase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the reference polypeptide.

As used herein, the term “gene” is defined to mean a polynucleotide (e.g., a DNA segment) that encodes a polypeptide. The term includes regions preceding and following the coding regions as well as any intervening sequences when present (e.g., introns) between individual coding segments (exons).

As used herein, the term “homologous genes” is defined to mean a pair of genes which correspond to each other and which are identical or similar to each other. The term encompasses genes that are separated by speciation (i.e., the development of new species) (e.g., orthologous genes), as well as genes that have been separated by genetic duplication (e.g., paralogous genes).

As used herein, the terms “ortholog” and “orthologous genes” are defined to mean genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, the terms “paralog” and “paralogous genes” are defined to mean genes that are related by duplication within a genome. Generally, paralogs tend to evolve into new functions, even though some functions are often related to the original one.

As used herein, the term “chromosomal integration” is defined to mean the process whereby an incoming sequence is introduced into the chromosome of a host cell. The homologous regions of the transforming DNA align with homologous regions of the chromosome. Subsequently, the sequence between the homology boxes is replaced by the incoming sequence in a double crossover (i.e., homologous recombination). In some embodiments, homologous sections of an inactivating chromosomal segment of a DNA construct align with the flanking homologous regions of the indigenous chromosomal region of a host cell chromosome. Subsequently, the indigenous chromosomal region is deleted by the DNA construct in a double crossover (i.e., homologous recombination).

As used herein, the term “homologous recombination” is defined to mean the exchange of DNA fragments between two DNA molecules or paired chromosomes at the site of identical or nearly identical nucleotide sequences. In some embodiments, chromosomal integration is homologous recombination.

As used herein, the term “isolated polypeptide” is defined to mean a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis).

The variant cytochrome P450 monooxygenase may be present within a cell, present in the cellular medium, or prepared in various forms, such as lysates or isolated preparations. As such, in some embodiments, the variant cytochrome P450 monooxygenase can be an isolated polypeptide.

As used herein, the term “substantially pure polypeptide” is defined to mean a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure variant cytochrome P450 monooxygenase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated variant cytochrome P450 monooxygenase is a substantially pure polypeptide composition.

As used herein, the term “improved enzyme property” is defined to mean a variant cytochrome P450 monooxygenase that exhibits an improvement in any enzyme property as compared to a reference cytochrome P450 monooxygenase. For the variant cytochrome P450 monooxygenase described herein, the comparison is generally made to the naturally occurring enzyme having cytochrome P450 monooxygenase activity, although in some aspects, the comparator cytochrome P450 monooxygenase polypeptide can be another variant cytochrome P450 monooxygenase. Enzyme properties for which improvement is desirable include, but are not limited to, enzymatic activity (which can be expressed in terms of percent conversion of the substrate), thermo stability, solvent stability, pH activity profile, cofactor requirements, refractoriness to inhibitors (e.g., substrate or product inhibition), or substrate specificity.

As used herein, the term “increased enzymatic activity” is defined to mean an improved property of the cytochrome P450 monooxygenase, which can be represented by an increase in specific activity (e.g., product produced/time/weight protein) or an increase in percent conversion of the substrate to the product (e.g., percent conversion of starting amount of substrate to product in a specified time period using a specified amount of cytochrome P450 monooxygenase) as compared to a reference cytochrome P450 monooxygenase enzyme.

Any property relating to enzyme activity may be affected, including the enzyme properties of Kₘ, Vₘₐₓ or k_{cat}, changes of which can lead to increased enzymatic activity. Improvements in enzyme activity can be from about 1.2 times the enzymatic activity of the corresponding cytochrome P450 monooxygenase enzyme, to as much as 2 times, 5 times, 10 times, 20 times, 25 times, 50 times or more enzymatic activity than the comparator cytochrome P450 monooxygenase. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, the term “conversion” is defined to mean the enzymatic conversion of the substrate(s) to the corresponding product(s). “Percent conversion” refers to the percent of the substrate that is converted to the product within a period of time under specified conditions. Thus, the “enzymatic activity” or “activity” of a cytochrome P450 monooxygenase can be expressed as “percent conversion” of the substrate to the product.

As used herein, the term “specificity” as used in reference to an biocatalyst or enzyme is defined to mean the discrimination of the biocatalyst for a substrate compound.

As used herein, the term “relative specificity” is defined to mean the specificity of a biocatalyst or enzyme for one substrate compound over another or other substrate compounds.

As used herein, the term “stringent hybridization conditions” is defined to mean hybridizing in 50% formamide at 5×SSC at a temperature of 42° C. and washing the filters in 0.2×SSC at 60° C. (1×SSC is 0.15M NaCl, 0.015M sodium citrate.) Stringent hybridization conditions also encompasses low ionic strength and high temperature for washing, for example 0.015 M sodium chloride/0.0015 M sodium citrate/0.1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As defined herein, the term “heterologous” polynucleotide or polypeptide is defined to mean any polynucleotide or polypeptide that is not naturally found in a host cell. As such, the term includes polynucleotides that are removed from a host cell, subjected to laboratory manipulation, and then reintroduced into a host cell. In some embodiments, the introduced polynucleotide expresses the heterologous polypeptide.

As used herein, the term “codon optimized” is defined to mean changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some aspects, the polynucleotides encoding the variant cytochrome P450 monooxygenase enzymes may be codon optimized for optimal production from the host organism selected for expression.

As used herein, the term “control sequence” is defined to include all components, which are necessary or advantageous for the expression of a polynucleotide and/or polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and where appropriate, translational stop signals. The control sequences may be provided with linkers for the purpose of introducing specific restriction sites facilitating ligation of the control sequences with the coding region of the nucleic acid sequence encoding a polypeptide.

As used herein, the term “operably linked” is defined to mean a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the term “promoter sequence” is defined to mean a nucleic acid sequence that is recognized by a host cell for expression of a polynucleotide of interest, such as a coding sequence or gene. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

As used herein, the term “suitable reaction conditions” is defined to mean those conditions in the reaction solution (e.g., ranges of enzyme loading, substrate loading, cofactor loading, temperature, pH, buffers, co-solvents, etc.) under which a cytochrome P450 monooxygenase of the present disclosure is capable of converting substrate compound to a product compound.

As used herein, the terms “microbial,” “microbial organism” or “microorganism” are defined to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

### Variant Cytochrome P450 Monooxygenase

The disclosure provides engineered polypeptides having cytochrome P450 monooxygenase activity, polynucleotides encoding these polypeptides, host cells containing the polynucleotides, and methods for using the polypeptides and host cells for the bioremediation of crude oil and components of crude oil. Where the description relates to polypeptides, it is to be understood that it also describes the polynucleotides encoding the polypeptides.

Cytochrome P450 monooxygenase catalyzes the oxidation of fatty acids, crude oil, and crude oil components. Cytochrome P450 monooxygenase can contain heme as a cofactor, and these enzymes can be utilized as an oxidase in electron transfer chains. Cytochrome P450 monooxygenase can be used in breakdown and metabolism of certain components in crude oil (e.g., dodecane).

The disclosure provides variant cytochrome P450 monooxygenase having improved activity for the breakdown and metabolism of components of crude oil. In one aspect, the variant cytochrome P450 monooxygenase can increase the growth rate of Bacillus megaterium when it utilizes crude oil or dodecane as a carbon source. Variants were made by site directed mutagenesis of certain amino acid positions in cytochrome P450 monooxygenase. These positions of cytochrome P450 monooxygenase include, for example, L20, P25, V26, L29, F42, R47, V48, T49, Y51, A74, L75, V78, A82, L86, F87, W96, H138, V178, F181, A184, L188, H236, M237, E252, R255, F261, A264, T260, I263, T268, A290, A295, A328, P329, A330, L353, M354, P382, S383, A384, I385, P386, Q387, F393, I401, F405, L437, and T438. For each amino acid position, variants of cytochrome P450 monooxygenase were made that changed to selected amino acid position to all 19 other amino acids. Pools of Variants for each position were screened for increased growth rate of Bacillus megaterium transformed with the variant cytochrome P450 monooxygenase. Clones with increased growth rates were selected, and the activity of the cytochrome P450 monooxygenase in the Bacillus megaterium was measured using crude oil, dodecane, or benzopyrene was a substrate. The reaction rate and products of the reaction were measured using GCMS. Six (6) variants showed increased growth rates of from 8% to 80% on dodecane and 12% to 68% on crude oil. Eight (8) additional variants also showed increased growth rates on either dodecane or crude oil.

The variants obtained were: F42W, H138A, L86N, V78W, L75N, T49S, T49A, F42T, F42V, F42C, F42G, F42I, F42R, and F42D. The amino acids F42 and T49 form part of the entry way or hatch for the entry of substrates and holding substrates in place by the cytochrome P450 monooxygenase. The amino acid L86 interacts with the heme group. The amino acid V78 is related to the binding pocket and the change V78W may make the binding pocket larger allowing larger substrates to be bound. The polynucleotide sequence and amino acid sequence some of the variants are shown below:

F42W
(SEQ ID NO: 1)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATGGGAGGCGCCTGGTCGTGTAACGCGCTACTTATCAAGTCAGCG
TCTAATTAAAGAAGCATGCGATGAATCACGCTTTGATAAAAACTTAAGTCAAGCGCT
TAAATTTGTACGTGATTTTGCAGGAGACGGGTTATTTACAAGCTGGACGCATGAAAA
AAATTGGAAAAAAGCGCATAATATCTTACTTCCAAGCTTCAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTACCGGAAGACATGACACGTTTAACGCTT
GATACAATTGGTCTTTGCGGCTTTAACTATCGCTTTAACAGCTTTTACCGAGATCAGC
CTCATCCATTTATTACAAGTATGGTCCGTGCACTGGATGAAGCAATGAACAAGCTGC
AGCGAGCAAATCCAGACGACCCAGCTTATGATGAAAACAAGCGCCAGTTTCAAGAA
GATATCAAGGTGATGAACGACCTAGTAGATAAAATTATTGCAGATCGCAAAGCAAG
CGGTGAACAAAGCGATGATTTATTAACGCATATGCTAAACGGAAAAGATCCAGAAA
CGGGTGAGCCGCTTGATGACGAGAACATTCGCTATCAAATTATTACATTCTTAATTG
CGGGACACGAAACAACAAGTGGTCTTTTATCATTTGCGCTGTATTTCTTAGTGAAAA
ATCCACATGTATTACAAAAAGCAGCAGAAGAAGCAGCACGAGTTCTAGTAGATCCT
GTTCCAAGCTACAAACAAGTCAAACAGCTTAAATATGTCGGCATGGTCTTAAACGA
AGCGCTGCGCTTATGGCCAACTGCTCCTGCGTTTTCCCTATATGCAAAAGAAGATAC
GGTGCTTGGAGGAGAATATCCTTTAGAAAAAGGCGACGAACTAATGGTTCTGATTCC
TCAGCTTCACCGTGATAAAACAATTTGGGGAGACGATGTGGAAGAGTTCCGTCCAG
AGCGTTTTGAAAATCCAAGTGCGATTCCGCAGCATGCGTTTAAACCGTTTGGAAACG
GTCAGCGTGCGTGTATCGGTCAGCAGTTCGCTCTTCATGAAGCAACGCTGGTACTTG
GTATGATGCTAAAACACTTTGACTTTGAAGATCATACAAACTACGAGCTGGATATTA
AAGAAACTTTAACGTTAAAACCTGAAGGCTTTGTGGTAAAAGCAAAATCGAAAAAA
ATTCCGCTTGGCGGTATTCCTTCACCTAGCACTGAACAGTCTGCTAAAAAAGTACGC
AAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA
(SEQ ID NO: 2)
MTIKEMPQPKTFGELKNLPLLNTDKPVQALMKIADELGEIFKWEAPGRVTRYLSSQRLIK
EACDESRFDKNLSQALKFVRDFAGDGLFTSWTHEKNWKKAHNILLPSFSQQAMKGYH
AMMVDIAVQLVQKWERLNADEHIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG
H138A
(SEQ ID NO: 3)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGCT
TAAATTTGTACGTGATTTTGCAGGAGACGGGTTATTTACAAGCTGGACGCATGAAAA
AAATTGGAAAAAAGCGCATAATATCTTACTTCCAAGCTTCAGTCAGCAGGCAATGA
AAGGCTATCATGCGATGATGGTCGATATCGCCGTGCAGCTTGTTCAAAAGTGGGAGC
GTCTAAATGCAGATGAGGCCATTGAAGTACCGGAAGACATGACACGTTTAACGCTT
GATACAATTGGTCTTTGCGGCTTTAACTATCGCTTTAACAGCTTTTACCGAGATCAGC
CTCATCCATTTATTACAAGTATGGTCCGTGCACTGGATGAAGCAATGAACAAGCTGC
AGCGAGCAAATCCAGACGACCCAGCTTATGATGAAAACAAGCGCCAGTTTCAAGAA
GATATCAAGGTGATGAACGACCTAGTAGATAAAATTATTGCAGATCGCAAAGCAAG
CGGTGAACAAAGCGATGATTTATTAACGCATATGCTAAACGGAAAAGATCCAGAAA
CGGGTGAGCCGCTTGATGACGAGAACATTCGCTATCAAATTATTACATTCTTAATTG
CGGGACACGAAACAACAAGTGGTCTTTTATCATTTGCGCTGTATTTCTTAGTGAAAA
ATCCACATGTATTACAAAAAGCAGCAGAAGAAGCAGCACGAGTTCTAGTAGATCCT
GTTCCAAGCTACAAACAAGTCAAACAGCTTAAATATGTCGGCATGGTCTTAAACGA
AGCGCTGCGCTTATGGCCAACTGCTCCTGCGTTTTCCCTATATGCAAAAGAAGATAC
GGTGCTTGGAGGAGAATATCCTTTAGAAAAAGGCGACGAACTAATGGTTCTGATTCC
TCAGCTTCACCGTGATAAAACAATTTGGGGAGACGATGTGGAAGAGTTCCGTCCAG
AGCGTTTTGAAAATCCAAGTGCGATTCCGCAGCATGCGTTTAAACCGTTTGGAAACG
GTCAGCGTGCGTGTATCGGTCAGCAGTTCGCTCTTCATGAAGCAACGCTGGTACTTG
GTATGATGCTAAAACACTTTGACTTTGAAGATCATACAAACTACGAGCTGGATATTA
AAGAAACTTTAACGTTAAAACCTGAAGGCTTTGTGGTAAAAGCAAAATCGAAAAAA
ATTCCGCTTGGCGGTATTCCTTCACCTAGCACTGAACAGTCTGCTAAAAAAGTACGC
AAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA
(SEQ ID NO: 4)
MTIKEMPQPKTFGELKNLPLLNTDKPVQALMKIADELGEIFKFEAPGRVTRYLSSQRLIK
EACDESRFDKNLSQALKFVRDFAGDGLFTSWTHEKNWKKAHNILLPSFSQQAMKGYH
AMMVDIAVQLVQKWERLNADEAIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG
L86N
(SEQ ID NO: 5)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGCT
TAAATTTGTACGTGATTTTGCAGGAGACGGGAATTTTACAAGCTGGACGCATGAAAA
AAATTGGAAAAAAGCGCATAATATCTTACTTCCAAGCTTCAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTACCGGAAGACATGACACGTTTAACGCTT
GATACAATTGGTCTTTGCGGCTTTAACTATCGCTTTAACAGCTTTTACCGAGATCAGC
CTCATCCATTTATTACAAGTATGGTCCGTGCACTGGATGAAGCAATGAACAAGCTGC
AGCGAGCAAATCCAGACGACCCAGCTTATGATGAAAACAAGCGCCAGTTTCAAGAA
GATATCAAGGTGATGAACGACCTAGTAGATAAAATTATTGCAGATCGCAAAGCAAG
CGGTGAACAAAGCGATGATTTATTAACGCATATGCTAAACGGAAAAGATCCAGAAA
CGGGTGAGCCGCTTGATGACGAGAACATTCGCTATCAAATTATTACATTCTTAATTG
CGGGACACGAAACAACAAGTGGTCTTTTATCATTTGCGCTGTATTTCTTAGTGAAAA
ATCCACATGTATTACAAAAAGCAGCAGAAGAAGCAGCACGAGTTCTAGTAGATCCT
GTTCCAAGCTACAAACAAGTCAAACAGCTTAAATATGTCGGCATGGTCTTAAACGA
AGCGCTGCGCTTATGGCCAACTGCTCCTGCGTTTTCCCTATATGCAAAAGAAGATAC
GGTGCTTGGAGGAGAATATCCTTTAGAAAAAGGCGACGAACTAATGGTTCTGATTCC
TCAGCTTCACCGTGATAAAACAATTTGGGGAGACGATGTGGAAGAGTTCCGTCCAG
AGCGTTTTGAAAATCCAAGTGCGATTCCGCAGCATGCGTTTAAACCGTTTGGAAACG
GTCAGCGTGCGTGTATCGGTCAGCAGTTCGCTCTTCATGAAGCAACGCTGGTACTTG
GTATGATGCTAAAACACTTTGACTTTGAAGATCATACAAACTACGAGCTGGATATTA
AAGAAACTTTAACGTTAAAACCTGAAGGCTTTGTGGTAAAAGCAAAATCGAAAAAA
ATTCCGCTTGGCGGTATTCCTTCACCTAGCACTGAACAGTCTGCTAAAAAAGTACGC
AAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA
(SEQ ID NO: 6)
MTIKEMPQPKTFGELKNLPLLNTDKPVQALMKIADELGEIFKFEAPGRVTRYLSSQRLIK
EACDESRFDKNLSQALKFVRDFAGDGNFTSWTHEKNWKKAHNILLPSFSQQAMKGYH
AMMVDIAVQLVQKWERLNADEHIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG
V78W
(SEQ ID NO: 7)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGCT
TAAATTTTGGCGTGATTTTGCAGGAGACGGGAATTTTACAAGCTGGACGCATGAAAA
AAATTGGAAAAAAGCGCATAATATCTTACTTCCAAGCTTCAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTACCGGAAGACATGACACGTTTAACGCTT
GATACAATTGGTCTTTGCGGCTTTAACTATCGCTTTAACAGCTTTTACCGAGATCAGC
CTCATCCATTTATTACAAGTATGGTCCGTGCACTGGATGAAGCAATGAACAAGCTGC
AGCGAGCAAATCCAGACGACCCAGCTTATGATGAAAACAAGCGCCAGTTTCAAGAA
GATATCAAGGTGATGAACGACCTAGTAGATAAAATTATTGCAGATCGCAAAGCAAG
CGGTGAACAAAGCGATGATTTATTAACGCATATGCTAAACGGAAAAGATCCAGAAA
CGGGTGAGCCGCTTGATGACGAGAACATTCGCTATCAAATTATTACATTCTTAATTG
CGGGACACGAAACAACAAGTGGTCTTTTATCATTTGCGCTGTATTTCTTAGTGAAAA
ATCCACATGTATTACAAAAAGCAGCAGAAGAAGCAGCACGAGTTCTAGTAGATCCT
GTTCCAAGCTACAAACAAGTCAAACAGCTTAAATATGTCGGCATGGTCTTAAACGA
AGCGCTGCGCTTATGGCCAACTGCTCCTGCGTTTTCCCTATATGCAAAAGAAGATAC
GGTGCTTGGAGGAGAATATCCTTTAGAAAAAGGCGACGAACTAATGGTTCTGATTCC
TCAGCTTCACCGTGATAAAACAATTTGGGGAGACGATGTGGAAGAGTTCCGTCCAG
AGCGTTTTGAAAATCCAAGTGCGATTCCGCAGCATGCGTTTAAACCGTTTGGAAACG
GTCAGCGTGCGTGTATCGGTCAGCAGTTCGCTCTTCATGAAGCAACGCTGGTACTTG
GTATGATGCTAAAACACTTTGACTTTGAAGATCATACAAACTACGAGCTGGATATTA
AAGAAACTTTAACGTTAAAACCTGAAGGCTTTGTGGTAAAAGCAAAATCGAAAAAA
ATTCCGCTTGGCGGTATTCCTTCACCTAGCACTGAACAGTCTGCTAAAAAAGTACGC
AAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA
(SEQ ID NO: 8)
MTIKEMPQPKTFGELKNLPLLNTDKPVQALMKIADELGEIFKFEAPGRVTRYLSSQRLIK
EACDESRFDKNLSQALKFWRDFAGDGNFTSWTHEKNWKKAHNILLPSFSQQAMKGYH
AMMVDIAVQLVQKWERLNADEHIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG
L75N
(SEQ ID NO: 9)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGA
ATAAATTTGTACGTGATTTTGCAGGAGACGGGAATTTTACAAGCTGGACGCATGAAA
AAAATTGGAAAAAAGCGCATAATATCTTACTTCCAAGCTTCAGTCAGCAGGCAATG
AAAGGCTATCATGCGATGATGGTCGATATCGCCGTGCAGCTTGTTCAAAAGTGGGA
GCGTCTAAATGCAGATGAGCATATTGAAGTACCGGAAGACATGACACGTTTAACGC
TTGATACAATTGGTCTTTGCGGCTTTAACTATCGCTTTAACAGCTTTTACCGAGATCA
GCCTCATCCATTTATTACAAGTATGGTCCGTGCACTGGATGAAGCAATGAACAAGCT
GCAGCGAGCAAATCCAGACGACCCAGCTTATGATGAAAACAAGCGCCAGTTTCAAG
AAGATATCAAGGTGATGAACGACCTAGTAGATAAAATTATTGCAGATCGCAAAGCA
AGCGGTGAACAAAGCGATGATTTATTAACGCATATGCTAAACGGAAAAGATCCAGA
AACGGGTGAGCCGCTTGATGACGAGAACATTCGCTATCAAATTATTACATTCTTAAT
TGCGGGACACGAAACAACAAGTGGTCTTTTATCATTTGCGCTGTATTTCTTAGTGAA
AAATCCACATGTATTACAAAAAGCAGCAGAAGAAGCAGCACGAGTTCTAGTAGATC
CTGTTCCAAGCTACAAACAAGTCAAACAGCTTAAATATGTCGGCATGGTCTTAAACG
AAGCGCTGCGCTTATGGCCAACTGCTCCTGCGTTTTCCCTATATGCAAAAGAAGATA
CGGTGCTTGGAGGAGAATATCCTTTAGAAAAAGGCGACGAACTAATGGTTCTGATTC
CTCAGCTTCACCGTGATAAAACAATTTGGGGAGACGATGTGGAAGAGTTCCGTCCA
GAGCGTTTTGAAAATCCAAGTGCGATTCCGCAGCATGCGTTTAAACCGTTTGGAAAC
GGTCAGCGTGCGTGTATCGGTCAGCAGTTCGCTCTTCATGAAGCAACGCTGGTACTT
GGTATGATGCTAAAACACTTTGACTTTGAAGATCATACAAACTACGAGCTGGATATT
AAAGAAACTTTAACGTTAAAACCTGAAGGCTTTGTGGTAAAAGCAAAATCGAAAAA
AATTCCGCTTGGCGGTATTCCTTCACCTAGCACTGAACAGTCTGCTAAAAAAGTACG
CAAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA
(SEQ ID NO: 10)
MTIKEMPQPKTFGELKNLPLLNTDKPVQALMKIADELGEIFKFEAPGRVTRYLSSQRLIK
EACDESRFDKNLSQANKFVRDFAGDGNFTSWTHEKNWKKAHNILLPSFSQQAMKGYH
AMMVDIAVQLVQKWERLNADEHIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG
T49S
(SEQ ID NO: 11)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAAGTCGCTACTTATCAAGTCAGCG
TCTAATTAAAGAAGCATGCGATGAATCACGCTTTGATAAAAACTTAAGTCAAGCGCT
TAAATTTGTACGTGATTTTGCAGGAGACGGGTTATTTACAAGCTGGACGCATGAAAA
AAATTGGAAAAAAGCGCATAATATCTTACTTCCAAGCTTCAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTACCGGAAGACATGACACGTTTAACGCTT
GATACAATTGGTCTTTGCGGCTTTAACTATCGCTTTAACAGCTTTTACCGAGATCAGC
CTCATCCATTTATTACAAGTATGGTCCGTGCACTGGATGAAGCAATGAACAAGCTGC
AGCGAGCAAATCCAGACGACCCAGCTTATGATGAAAACAAGCGCCAGTTTCAAGAA
GATATCAAGGTGATGAACGACCTAGTAGATAAAATTATTGCAGATCGCAAAGCAAG
CGGTGAACAAAGCGATGATTTATTAACGCATATGCTAAACGGAAAAGATCCAGAAA
CGGGTGAGCCGCTTGATGACGAGAACATTCGCTATCAAATTATTACATTCTTAATTG
CGGGACACGAAACAACAAGTGGTCTTTTATCATTTGCGCTGTATTTCTTAGTGAAAA
ATCCACATGTATTACAAAAAGCAGCAGAAGAAGCAGCACGAGTTCTAGTAGATCCT
GTTCCAAGCTACAAACAAGTCAAACAGCTTAAATATGTCGGCATGGTCTTAAACGA
AGCGCTGCGCTTATGGCCAACTGCTCCTGCGTTTTCCCTATATGCAAAAGAAGATAC
GGTGCTTGGAGGAGAATATCCTTTAGAAAAAGGCGACGAACTAATGGTTCTGATTCC
TCAGCTTCACCGTGATAAAACAATTTGGGGAGACGATGTGGAAGAGTTCCGTCCAG
AGCGTTTTGAAAATCCAAGTGCGATTCCGCAGCATGCGTTTAAACCGTTTGGAAACG
GTCAGCGTGCGTGTATCGGTCAGCAGTTCGCTCTTCATGAAGCAACGCTGGTACTTG
GTATGATGCTAAAACACTTTGACTTTGAAGATCATACAAACTACGAGCTGGATATTA
AAGAAACTTTAACGTTAAAACCTGAAGGCTTTGTGGTAAAAGCAAAATCGAAAAAA
ATTCCGCTTGGCGGTATTCCTTCACCTAGCACTGAACAGTCTGCTAAAAAAGTACGC
AAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA
(SEQ ID NO: 12)
MTIKEMPQPKTFGELKNLPLLNTDKPVQALMKIADELGEIFKFEAPGRVSRYLSSQRLIK
EACDESRFDKNLSQALKFVRDFAGDGLFTSWTHEKNWKKAHNILLPSFSQQAMKGYH
AMMVDIAVQLVQKWERLNADEHIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG

Accordingly, the present disclosure can provide a variant cytochrome P450 monooxygenase polypeptide capable of components of crude oil with an improved enzyme property as compared to a reference enzyme, which can be a naturally occurring cytochrome P450 monooxygenase. In some aspects, the reference enzyme is encoded by SEQ ID NO: 13.

(SEQ ID NO: 13)
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGCT
TAAATTTGTACGTGATTTTGCAGGAGACGGGTTATTTACAAGCTGGACGCATGAAAA
AAATTGGAAAAAAGCGCATAATATCTTACTTCCAAGCTTCAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTACCGGAAGACATGACACGTTTAACGCTT
GATACAATTGGTCTTTGCGGCTTTAACTATCGCTTTAACAGCTTTTACCGAGATCAGC
CTCATCCATTTATTACAAGTATGGTCCGTGCACTGGATGAAGCAATGAACAAGCTGC
AGCGAGCAAATCCAGACGACCCAGCTTATGATGAAAACAAGCGCCAGTTTCAAGAA
GATATCAAGGTGATGAACGACCTAGTAGATAAAATTATTGCAGATCGCAAAGCAAG
CGGTGAACAAAGCGATGATTTATTAACGCATATGCTAAACGGAAAAGATCCAGAAA
CGGGTGAGCCGCTTGATGACGAGAACATTCGCTATCAAATTATTACATTCTTAATTG
CGGGACACGAAACAACAAGTGGTCTTTTATCATTTGCGCTGTATTTCTTAGTGAAAA
ATCCACATGTATTACAAAAAGCAGCAGAAGAAGCAGCACGAGTTCTAGTAGATCCT
GTTCCAAGCTACAAACAAGTCAAACAGCTTAAATATGTCGGCATGGTCTTAAACGA
AGCGCTGCGCTTATGGCCAACTGCTCCTGCGTTTTCCCTATATGCAAAAGAAGATAC
GGTGCTTGGAGGAGAATATCCTTTAGAAAAAGGCGACGAACTAATGGTTCTGATTCC
TCAGCTTCACCGTGATAAAACAATTTGGGGAGACGATGTGGAAGAGTTCCGTCCAG
AGCGTTTTGAAAATCCAAGTGCGATTCCGCAGCATGCGTTTAAACCGTTTGGAAACG
GTCAGCGTGCGTGTATCGGTCAGCAGTTCGCTCTTCATGAAGCAACGCTGGTACTTG
GTATGATGCTAAAACACTTTGACTTTGAAGATCATACAAACTACGAGCTGGATATTA
AAGAAACTTTAACGTTAAAACCTGAAGGCTTTGTGGTAAAAGCAAAATCGAAAAAA
ATTCCGCTTGGCGGTATTCCTTCACCTAGCACTGAACAGTCTGCTAAAAAAGTACGC
AAAAAGGCAGAAAACGCTCATAATACGCCGCTGCTTGTGCTATACGGTTCAAATAT
GGGAACAGCTGAAGGAACGGCGCGTGATTTAGCAGATATTGCAATGAGCAAAGGAT
TTGCACCGCAGGTCGCAACGCTTGATTCACACGCCGGAAATCTTCCGCGCGAAGGA
GCTGTATTAATTGTAACGGCGTCTTATAACGGTCATCCGCCTGATAACGCAAAGCAA
TTTGTCGACTGGTTAGACCAAGCGTCTGCTGATGAAGTAAAAGGCGTTCGCTACTCC
GTATTTGGATGCGGCGATAAAAACTGGGCTACTACGTATCAAAAAGTGCCTGCTTTT
ATCGATGAAACGCTTGCCGCTAAAGGGGCAGAAAACATCGCTGACCGCGGTGAAGC
AGATGCAAGCGACGACTTTGAAGGCACATATGAAGAATGGCGTGAACATATGTGGA
GTGACGTAGCAGCCTACTTTAACCTCGACATTGAAAACAGTGAAGATAATAAATCTA
CTCTTTCACTTCAATTTGTCGACAGCGCCGCGGATATGCCGCTTGCGAAAATGCACG
GTGCGTTTTCAACGAACGTCGTAGCAAGCAAAGAACTTCAACAGCCAGGCAGTGCA
CGAAGCACGCGACATCTTGAAATTGAACTTCCAAAAGAAGCTTCTTATCAAGAAGG
AGATCATTTAGGTGTTATTCCTCGCAACTATGAAGGAATAGTAAACCGTGTAACAGC
AAGGTTCGGCCTAGATGCATCACAGCAAATCCGTCTGGAAGCAGAAGAAGAAAAAT
TAGCTCATTTGCCACTCGCTAAAACAGTATCCGTAGAAGAGCTTCTGCAATACGTGG
AGCTTCAAGATCCTGTTACGCGCACGCAGCTTCGCGCAATGGCTGCTAAAACGGTCT
GCCCGCCGCATAAAGTAGAGCTTGAAGCCTTGCTTGAAAAGCAAGCCTACAAAGAA
CAAGTGCTGGCAAAACGTTTAACAATGCTTGAACTGCTTGAAAAATACCCGGCGTGT
GAAATGAAATTCAGCGAATTTATCGCCCTTCTGCCAAGCATACGCCCGCGCTATTAC
TCGATTTCTTCATCACCTCGTGTCGATGAAAAACAAGCAAGCATCACGGTCAGCGTT
GTCTCAGGAGAAGCGTGGAGCGGATATGGAGAATATAAAGGAATTGCGTCGAACTA
TCTTGCCGAGCTGCAAGAAGGAGATACGATTACGTGCTTTATTTCCACACCGCAGTC
AGAATTTACGCTGCCAAAAGACCCTGAAACGCCGCTTATCATGGTCGGACCGGGAA
CAGGCGTCGCGCCGTTTAGAGGCTTTGTGCAGGCGCGCAAACAGCTAAAAGAACAA
GGACAGTCACTTGGAGAAGCACATTTATACTTCGGCTGCCGTTCACCTCATGAAGAC
TATCTGTATCAAGAAGAGCTTGAAAACGCCCAAAGCGAAGGCATCATTACGCTTCAT
ACCGCTTTTTCTCGCATGCCAAATCAGCCGAAAACATACGTTCAGCACGTAATGGAA
CAAGACGGCAAGAAATTGATTGAACTTCTTGATCAAGGAGCGCACTTCTATATTTGC
GGAGACGGAAGCCAAATGGCACCTGCCGTTGAAGCAACGCTTATGAAAAGCTATGC
TGACGTTCACCAAGTGAGTGAAGCAGACGCTCGCTTATGGCTGCAGCAGCTAGAAG
AAAAAGGCCGATACGCAAAAGACGTGTGGGCTGGGTAA

In some aspects, the reference enzyme corresponds to an amino acid sequence of SEQ ID NO: 14.

(SEQ ID NO: 14)
MTIKEMPQPKTFGELKNLPLLNTDKPVQALMKIADELGEIFKFEAPGRVTRYLSSQRLIK
EACDESRFDKNLSQALKFVRDFAGDGLFTSWTHEKNWKKAHNILLPSFSQQAMKGYH
AMMVDIAVQLVQKWERLNADEHIEVPEDMTRLTLDTIGLCGFNYRFNSFYRDQPHPFIT
SMVRALDEAMNKLQRANPDDPAYDENKRQFQEDIKVMNDLVDKIIADRKASGEQSDD
LLTHMLNGKDPETGEPLDDENIRYQIITFLIAGHETTSGLLSFALYFLVKNPHVLQKAAEE
AARVLVDPVPSYKQVKQLKYVGMVLNEALRLWPTAPAFSLYAKEDTVLGGEYPLEKG
DELMVLIPQLHRDKTIWGDDVEEFRPERFENPSAIPQHAFKPFGNGQRACIGQQFALHEA
TLVLGMMLKHFDFEDHTNYELDIKETLTLKPEGFVVKAKSKKIPLGGIPSPSTEQSAKKV
RKKAENAHNTPLLVLYGSNMGTAEGTARDLADIAMSKGFAPQVATLDSHAGNLPREG
AVLIVTASYNGHPPDNAKQFVDWLDQASADEVKGVRYSVFGCGDKNWATTYQKVPAF
IDETLAAKGAENIADRGEADASDDFEGTYEEWREHMWSDVAAYFNLDIENSEDNKSTL
SLQFVDSAADMPLAKMHGAFSTNVVASKELQQPGSARSTRHLEIELPKEASYQEGDHL
GVIPRNYEGIVNRVTARFGLDASQQIRLEAEEEKLAHLPLAKTVSVEELLQYVELQDPVT
RTQLRAMAAKTVCPPHKVELEALLEKQAYKEQVLAKRLTMLELLEKYPACEMKFSEFI
ALLPSIRPRYYSISSSPRVDEKQASITVSVVSGEAWSGYGEYKGIASNYLAELQEGDTITC
FISTPQSEFTLPKDPETPLIMVGPGTGVAPFRGFVQARKQLKEQGQSLGEAHLYFGCRSP
HEDYLYQEELENAQSEGIITLHTAFSRMPNQPKTYVQHVMEQDGKKLIELLDQGAHFYI
CGDGSQMAPAVEATLMKSYADVHQVSEADARLWLQQLEEKGRYAKDVWAG

It is to be understood that various orthologs and paralogs, including orthologs and paralogs of SEQ ID NOs: 13 and/or 14, can be used as the reference enzyme.

The variant cytochrome P450 monooxygenase can have increased specificity for certain components of crude oil (e.g., dodecane) as compared to a reference polypeptide, e.g., SEQ ID NO: 14. The variant cytochrome P450 monooxygenase polypeptide can have lower specificity for substrates as compared to the reference polypeptide, e.g., SEQ ID NO: 14. The variant cytochrome P450 monooxygenase polypeptide can also have increased specificity or activity as compared to a reference polypeptide.

The advantageous properties of the variant cytochrome P450 monooxygenase disclosed herein can be associated with amino acid substitutions at residue positions corresponding to F42, T49, L75, V78, L86, and H138, where the amino acid substitutions and residue positions are with respect to the primary translation product starting at the methionine initiation codon (SEQ ID NO: 14). Without being bound by theory, amino acid substitutions at the foregoing residue positions affect the activity of cytochrome P450 monooxygenase. While the residue positions correspond to the reference sequence of SEQ ID NO:14, it is to be understood that equivalent residue positions can be identified in other related enzymes that have structural similarity to the reference sequence of SEQ ID NO: 14, including various orthologs and paralogs. The equivalent positions are readily determined by alignment of a target sequence with the reference sequence using sequence alignment software, particularly using optimal alignment of the target sequence with the reference sequence as described herein and as is well known in the art. In some aspects, the reference sequence comprises a polypeptide comprising a canonical or consensus sequence.

In some aspects, the variant cytochrome P450 monooxygenase has at least 60%, 65%, 70%, 75%, 80%, 85%, 90%, 91%, 92%, 93%, 94%, 95%, 96%, 97%, 98%, 99% or more amino acid sequence identity to the reference sequence of SEQ ID NO: 15 and having at least two amino acid substations at residue positions corresponding to: F42, T49, L75, V78, L86, and H138 of SEQ ID NO: 15.

In some aspects, variant cytochrome P450 monooxygenase has at least 60%, 65%, 70%, 75%, 80%, 85%, 90%, 91%, 92%, 93%, 94%, 95%, 96%, 97%, 98%, 99% or more amino acid sequence identity to the reference sequence of SEQ ID NO: 15 and having at least three amino acid substitutions at residue positions F42, T49, L75, V78, L86, and H138 of SEQ ID NO: 15.

In some aspects of the variant cytochrome P450 monooxygenase described herein, the amino acid at residue position:
F42 is selected from W, T, V, C, G, I, R, D;
T49 is selected from S, A;
L75 is selected from N;
V78 is selected from W;
L86 is selected from N;
H138 is selected from A.

Cytochrome P450 monooxygenase is a super family of enzymes which have a heme-iron center with highly conserved amino acid residues. The bacterial members of the family are highly conserved, and the variants disclosed herein can be made in any number of these other highly conserved bacterial cytochrome P450 monooxygenases. For example, bacterial family members can be altered with one or more of the above variants for the positions analogous to F42, T49, L75, V78, L86, and H138 in the homologs and paralogs. The following bacterial family members can be altered with one or more of the above variants including, for example, Uniprot Accession Numbers A0A0N1NK20, A0A2U1F6T2, A0A2U1FG79, A0A7Y0KKY1, A0A1H1LC32, A0A814PDN7, K8PK16, A0A1H0Q8Y0, A0A0Q6YRJ6, E0MRC9, A0A1M6L688, A0A2T4U4T2, A0A0J6CP59, W4Q5P2, A0A562USA7, A0A850H634, A0A1G8JEE9, A0A558BTC6, A0A0D1XUB7, A0A0USB146, A0A1Q3HUX8, A0A3N0C1C8, A0A554VTT5, V4PI21, A0A6L7HJT4, Q81BF4, Q65GU6, A8FDP1, A0A5C0WN88, A0A3A1QVK4, A0A2A8SE92, A0A2A8SEL8, A0A1Q9PR93, A0A176JB29, A0A176JB54, O08336, O08394, A0A4Q5NSI4, A0A0A1PYL7, A0A4Y9PEU1, Q89R90, A0A1M7SY96, A0A1B1ULG6, A0A508TAY6, A0A0D7P697, A0A075R3F8, A0A075R4D1, A0A7C9RFC1, A0A0K1E5F6, A0A231RDS7, A0A0Q0UDZ8, A0A7W7C9J0, A0A1L9B8J2, A0A1L9BDT1, A0A1L9BKT0, H8H2P3, A0A239VCC0, A0A402B865, A0A401ZGJ9, A0A402ALY8, A0A5J4KLX3, A0A814WFX7, A0A1T4L7J1, A0A7L4YPV3, Q2NDX9, A3WHN9, A0A109LQ32, A0A3L7JUP2, 18AFG7, A0A1W2E560, A0A372J101, 10JHR8, A0A4Z0H3A7, L5N6V8, A0A0B0D3P2, A0A1H210Y6, A0A839DU69, A0A0P6YF42, A0A0C2W4W6, A0A4V3AZY5, K6XDJ3, D6TEN2, D6U5T4, A0A718EPC9, A0A8J3HXT7, A0A8J3MS10, A0A4P6JTA2, W5VZN0, A0A7W9KGI8, W7STP7, A0A6M8HLN3, A0Y8J6, A0A1I6I615, A0A250ICW3, A0A1H7VPN6, A0A438ALF8, A0A1I3V171, A0A1X7N5G7, A0A1C2DNC7, A0A7R7CHA3, A0A7R7HBW7, A0A084H279, A0A7X2IX52, A0A615WMU1, A0A4Q4XGQ8, A0A0T1WCC9, D2VNQ7, C8X7L1, C8XB87, K6EB81, A0A2N5HPB1, A0A0U1NXC3, A0A444LMM31, A0A317NHG4, A0A5C4VXA4, A0A4Q5J0G2, A0A840W1X1, A0A7W8A5T1, I9C4C6, A0A8J6MJ11, A0A542ZFV2, A0A4Q2LDG6, A0A168JDG6, A0A0D5NK70, A0A172ZMF6, H3SM19, H3SPT4, A0A2Z2KXV1, A0A5D0CY51, A0A7X3K123, A0A090ZLC1, A0A1E3L8V5, A0A371PKP2, A0A7W5B281, E3E5G2, A0A1R1F2L4, A0A2W1LR86, A0A0M1P527, A0A269W044, A0A2S0U9W6, A0A0M2VXT6, A0A1R1CDV3, F5L1Y9, A0A0D3V954, V9GFP3, A0A1B8UT13, A0A1B8UVF4, A0A0Q4RGX9, A0A0Q7SIK1, A0A069D714, A0A172TQ32, A0A081PA63, A0A1X7HBC5, A0A559J184, A0A559J476, A0A1U7C1L0, A0A0K9GTW0, A0A1B3XTB8, A0A4D7QFV0, A0A1Q2L111, A0A0U2N411, A0A0A2VBX0, A0A0A5I4I0, A0A0A2TAG9, A0A844Z6B2, A0A1X7DEM9, A0A0M0KW01, D5DYE1, P14779, A0A5R9FCR5, A0A1I4TU36, A0A511DIP7, A0A848DPP3, F4D1V6, A0A0P0SI46, A0A263DQR4, A0A1M6Q4K4, A0A419HIJ5, A0A8J3IKA0, A0A512NGP7, A0A1I1CL73, A0A0Q7XLC3, A0A6A7MDF0, A0A1J6X0V3, A0A0M0G514, A0A0P6W4C2, A0A814ISZ5, A0A814UU23, A0A816EL15, A0A818NJ81, A0A254NKB3, A0A840NP20, A0A1V2QSA7, A0A0F0HJ47, A0A323TBE0, L0DPU7, A0A1Z2KV95, A0A2G1XMP2, A0A7M2SKC6, A0A7M2SX44, A0A6G9GV75, A0A3S9PQ10, A0A3S9PS68, A0A7W7LP01, C9YVP1, A0A2P2GQD8, A0A1H0TJ78, A0A1H0V9Z1, A0A101UDY9, A0A6H0CES5, D6KF56, A0A1K2FPM7, A0A2G9DLZ7, A0A0G3URC8, A0A0X3WPA3, A0A1B9F1J9, A0A7L4XXE3, A0A7Y0B813, A0A7L5SMI3, A0A7K3GIE5, A0A3N5ABX1, A0A150VL66, A0A5B7V7E4, A0A5N8X984, A0A505DRY4, D2B0C9, A0A6P0CDJ4, A0A163YBJ0, A0A402A324, A0A077M0S6, A0A1H3XU53, A0A364KIP5, A0A328VIS3, A0A326UAP9, A0A0S3Q061, A0A431TEI8, A0A0H2MDJ0, E6V9D1, A0A519FE23, A0A1G6QNM8, A0A244EDG1, A0A244EIR3, A0A429WNI3, A0A126ZCQ6, A0A7G8W3T3, A0A6P2E477, A0A6P2E8C6, A0A115ULZ8, A0A5C8PN17, and A0A2T4VPV1.

Cytochrome P450 monooxygenase homologs with uniprot accession numbers A0A0M0KW01 (Priestia koreensis), A0A0B0D3P2 (Halobacillus sp. BBL2006), 008394 (Bacillus subtilis), A0A1H7VPN6 (Mesobacillus persicus), A0A1K2FPM7 (Streptomyces sp. F-1), and A0A839DU69 (Halosaccharopolyspora lacisalsi) have RMSD scores for alphafold structures of about 2 or less. Homologs A0A0M0KW01 (Priestia koreensis) RMSD=1.00, A0A0B0D3P2 (Halobacillus sp. BBL2006) RMSD=1.22, and 008394 (Bacillus subtilis) RMSD=1.53, have the same amino acids as SEQ ID NO: 14 at positions F42, T49, L75, V78, L86 and H138, or the homolog has a variant amino acid at one of these positions that has been identified as improving activity of the cytochrome P450 monooxygenase. Homolog A0A0M0KW01 (Priestia koreensis) has all six amino acids F42, T49, L75, V78, L86, and H138; homolog A0A0B0D3P2 (Halobacillus sp. BBL2006) has 4 amino acids F42, L75, V78, and L86, and the other two positions are S49 and A138 which are the same as the improvement variants for these positions; and homolog O08394 (Bacillus subtilis) has 4 amino acids T49, L75, V78, and L86, and the other two positions are I42 and A138 which are the same as improvement variants for these positions. Homolog A0A1H7VPN6 (Mesobacillus persicus) RMSD=1.47, has amino acids T49, L75, V78, and L86, and M42 which is conservative amino acid change from the improvement variants F42V, F42G, and F42I, and S138. This homolog may be engineered to change the 42 position to Tryptophan (M42W) and/or the 138 position to Alanine (S138A). Homolog A0A1K2FPM7 (Streptomyces sp. F-1) RMSD=2.15, L75, V78, and L86, and R42 which is an improvement variant for this position, 149 which is a conservative amino acid change from the improvement variant for this position, and T138. This homolog may be engineered to change the 42 position to Tryptophan (R42W) and/or the 49 position to Alanine (I49A) and/or the 138 position to Alanine (T138A). Homolog A0A839DU69 (Halosaccharopolyspora lacisalsi) RMSD=2.10, has amino acids L75 and L86, V42 and A138 which are improvement variants for these positions, 149 which is a conservative amino acid change from the improvement variant for this position, and 178. This homolog may be engineered to change the 42 position to Tryptophan (V42W), and/or the 49 position to Alanine (149A), and/or the 78 position to Tryptophan (178W).

The following cytochrome P450 monooxygenase homologs are the same at positions F42, T49, L75, V78, L86 and H138 for three to five amino acids, and the other positions have either an improvement variant or an amino acid that is a conservative change from an improvement variant, uniprot accession number: A0A176JB54 (Bacillus sp. SJS) F42, T49, L75, V78, L86, and A138; A0A0M0G514 (Paenibacillus sp. CAA11) F42, T49, L75, V78, L86, and V138; A0A3S9PS68 (Streptomyces luteoverticillatus) T49, L75, V78, L86, and L42 and A138; 18AFG7 (Fictibacillus macauensis) L75, V78, L86, H138, and S49 and M42; A0A168JDG6 (Paenibacillus antarcticus) L75, V78, L86, H138, and N42 and N49; A0A1B8UT13 (Paenibacillus sp. KS1) L75, V78, L86, H138, and L49 and M42; A0A2A8SEL8 (Bacillus sp. AFS018417) T49, V78, L86, H138, and L42 and M75; A0A317NHG4 (Nocardia neocaledoniensis) L75, V78, L86, and V42, A138 and M49; F5LIY9 (Paenibacillus sp. HGF7) L75, V78, L86, and L42, Q49, and I138; A0A558BTC6 (Amycolatopsis rhizosphaerae) L75, V78, L86, and L42, M49, and V138; A0A5C4VXA4 (Nocardioides albidus) L75, V78, L86, and L42, L49, and V138; L0DPU7 (Singulisphaera acidiphila) L75, V78, L86, and L42, L49, and V138; D2VNQ7 (Naegleria gruberi) L75, V78, L86, and M42, L49, and V138. The foregoing homologs can be improved by changing one or more of the six positions (amino acid positions 42, 49, 75, 78, 86 and 138) to the following: position 42 is selected from F, W, T, V, C, G, I, R, D; position 49 is selected from T, S, A; position 75 is selected from L or N; 78 is selected from V or W; position 86 is selected from L or N; and 138 is selected from H or A.

The following cytochrome P450 monooxygenase homologs are the same at positions F42, T49, L75, V78, L86 and H138 for five amino acids, uniprot accession number: A0A3L7JUP2 (Falsibacillus albus) F42, L75, V78, L86, H138, and 149; A0A559J476 (Paenibacillus vietnamensis) F42, T49, L75, V78, L86, and T138. The foregoing homologs can be improved by changing one or more of the six positions (amino acid positions 42, 49, 75, 78, 86 and 138) to the following: position 42 is selected from F, W, T, V, C, G, I, R, D; position 49 is selected from T, S, A; position 75 is selected from L or N; 78 is selected from V or W; position 86 is selected from L or N; and 138 is selected from H or A.

The following cytochrome P450 monooxygenase homologs are the same at positions F42, L75, V78, and L86, and have the improvement variant S49, uniprot accession number: A0A0A51410 (Pontibacillus halophilus) F42, L75, V78, L86, S49, and T138. The following cytochrome P450 monooxygenase homologs are the same at positions F42, L75, V78, and L86, and have the improvement variant S49, and S138, uniprot accession number: A0A4V3AZY5 (Jeotgalibacillus sp. S-D1); A0A0A2VBX0 (Pontibacillus chungwhensis); A0A0U5B146 (Aneurinibacillus soli); A0A0Q4RGX9 (Paenibacillus sp. Leaf72); A0A0U1NXC3 (Neobacillus massiliamazoniensis). The following cytochrome P450 monooxygenase homologs are the same at positions F42, L75, V78, and L86, and have the improvement variant S49, and E138, uniprot accession number: A0A084H279 (Metabacillus indicus); A0A0C2W4W6 (Jeotgalibacillus campisalis); A0A0J6CP59 (Alkalihalobacillus macyae); A0A0P6W4C2 (Rossellomorea vietnamensis); A0A0U2N4I1 (Planococcus rifietoensis); A0A1H3XU53 (Thalassobacillus cyri); A0A5C0WN88 (Bacillus safensis); A8FDP1 (Bacillus pumilus); I0JHR8 (Halobacillus halophilus); L5N6V8 (Halobacillus sp. BAB-2008); A0A5R9FCR5 (Pseudalkalibacillus caeni). The homolog A0A7L5SMI3 (Streptomyces sp. Rer75) T49, L75, L86, the improvement variant amino acids V42 and A138, and 178. The foregoing homologs can be improved by changing one or more of the six positions (amino acid positions 42, 49, 75, 78, 86 and 138) to the following: position 42 is selected from F, W, T, V, C, G, I, R, D; position 49 is selected from T, S, A; position 75 is selected from L or N; 78 is selected from V or W; position 86 is selected from L or N; and 138 is selected from H or A.

The following cytochrome P450 monooxygenase homologs are the same at positions T49, L75, V78, and L86, have a conservative amino acid change from the improvement variant A42, and have S138 or T138, uniprot accession number: A0A150VL66 (Streptomyces sp. WAC04657), A0A1B9F1J9 (Streptomyces sp. PTY08712), A0A6H0CES5 (Streptomyces sp. DSM 40868), A0A7K3GIE5 (Streptomyces sp. SID5789), A0A7Y0B813 (Streptomyces sp. R302), Q65GU6 (Bacillus licheniformis), A0A364K1P5 (Thermoflavimicrobium daqui). The following cytochrome P450 monooxygenase homologs are the same at positions T49, L75, V78, and L86, have a conservative amino acid change from the improvement variant A42, and have E138, uniprot accession number: A0A1J6X0V3 (Rossellomorea aquimaris), A0A3A1QVK4 (Bacillus salacetis), A0A8J3HXT7 (Ktedonospora formicarum), D6U5T4 (Ktedonobacter racemifer), A0A323TBE0 (Salipaludibacillus keqinensis), A0A7X2IX52 (Metabacillus lacus). The following cytochrome P450 monooxygenase homolog is the same at positions T49, L75, V78, and L86, have a conservative amino acid change from the improvement variant A42, and has R138, uniprot accession number: A0A2P2GQD8 (Streptomyces showdoensis). The following cytochrome P450 monooxygenase homolog is the same at positions T49, L75, V78, and L86, have a conservative amino acid change from the improvement variant W42, and has P138, uniprot accession number: A0A6G9GV75 (Streptomyces liangshanensis). The foregoing homologs can be improved by changing one or more of the six positions (amino acid positions 42, 49, 75, 78, 86 and 138) to the following: position 42 is selected from F, W, T, V, C, G, I, R, D; position 49 is selected from T, S, A; position 75 is selected from L or N; 78 is selected from V or W; position 86 is selected from L or N; and 138 is selected from H or A.

The following cytochrome P450 monooxygenase homolog is the same at positions L75, V78, and L86, have the improvement variant S49 or A49, a conservative amino acid change from the improvement variant A42, and has R138, uniprot accession number: A0A075R3F8 (Brevibacillus laterosporus), A0A075R4D1 (Brevibacillus laterosporus), A0A4Q2LDG6 (Paenibacillaceae bacterium). The following cytochrome P450 monooxygenase homolog is the same at positions L75, V78, and L86, have the improvement variant 142, a conservative amino acid change from the improvement variant A49, and has P138 or N138, uniprot accession number: A0A7L4XXE3 (Streptomyces sp. QHH-9511), Q81BF4 (Bacillus cereus). The following cytochrome P450 monooxygenase homolog is the same at positions L75, V78, and L86, has the improvement variant A138, a conservative amino acid change from the improvement variant A42, and has E49, uniprot accession number: A0A269W044 (Paenibacillus sp. 7541). The following cytochrome P450 monooxygenase homolog is the same at positions L75, V78, and L86, have a conservative amino acid change from the improvement variant A49 and S49, and has S138, uniprot accession number: A0A1E3L8V5 (Paenibacillus nuruki), A0A5D0CY51 (Paenibacillus faecis), H3SPT4 (Paenibacillus dendritiformis). The following cytochrome P450 monooxygenase homolog is the same at positions L75, V78, and L86, have a conservative amino acid change from the improvement variant A42 and A49, and has E138, uniprot accession number: A0A5J4KLX3 (Dictyobacter vulcani), A0A6A7MDF0 (Rhodospirillales bacterium), A0A718EPC9 (Ktedonobacteria bacterium brp13), K8PK16 (Afipia broomeae), A0A6L7HJT4 (Bacillus anthracis), A0A6P0CDJ4 (Sulfitobacter sediminilitoris), Q89R90 (Bradyrhizobium diazoefficiens). The following cytochrome P450 monooxygenase homolog is the same at positions L75, V78, and L86, have a conservative amino acid change from the improvement variant A42 and A49, and has P138, uniprot accession number: C9YVP1 (Streptomyces scabiei), D6KF56 (Streptomyces sp. e14). The following cytochrome P450 monooxygenase homolog is the same at positions L75, V78, and L86, have a conservative amino acid change from the improvement variant A42 and A49, and has F138, uniprot accession number: A0A254NKB3 (Saccharibacillus sp. 023). The following cytochrome P450 monooxygenase homolog is the same at positions L75, V78, and L86, have a conservative amino acid change from the improvement variant A42 and A49, and has R138, uniprot accession number: A0A840NP20 (Saccharopolyspora gloriosae). The foregoing homologs can be improved by changing one or more of the six positions (amino acid positions 42, 49, 75, 78, 86 and 138) to the following: position 42 is selected from F, W, T, V, C, G, I, R, D; position 49 is selected from T, S, A; position 75 is selected from L or N; 78 is selected from V or W; position 86 is selected from L or N; and 138 is selected from H or A.

The following cytochrome P450 monooxygenase homolog is the same at positions L75 and L86, have a conservative amino acid change from V78, the improvement variant V42 or I42, a conservative amino acid change from improvement variant A49, and the improvement variant A138 or a conservative amino acid change from A138, uniprot accession number: A0A1V2QSA7 (Saccharothrix sp. ALI-22-I), A0A077M0S6 (Tetrasphaera japonica T1-X7), 19C4C6 (Novosphingobium sp. Rr 2-17). The foregoing homologs can be improved by changing one or more of the six positions (amino acid positions 42, 49, 75, 78, 86 and 138) to the following: position 42 is selected from F, W, T, V, C, G, I, R, D; position 49 is selected from T, S, A; position 75 is selected from L or N; 78 is selected from V or W; position 86 is selected from L or N; and 138 is selected from H or A.

In some aspects, the variant cytochrome P450 monooxygenase has at least 60%, 65%, 70%, 75%, 80%, 85%, 90%, 91%, 92%, 93%, 94%, 95%, 96%, 97%, 98%, 99% or more amino acid sequence identity to the reference sequence of SEQ ID NO: 14 and at least the following amino acid substitutions:
F42W, H138A, L86N, V78W, L75N, T49S, T49A, F42T, F42V, F42C, F42G, F42I, F42R, and F42D.

In an aspect, the cytochrome P450 monooxygenase includes, for example, the following enzymes from different species (e.g., homologs). The P450 enzymes in the following list are identified by Uniprot Accession number (gene name): A0A069D714 (TCA2_0022), A0A075R3F8 (BRLA_c016970), A0A075R4D1 (BRLA_c023790), A0A077M0S6 (cypD BN12_1110006 BN12_4320005), A0A081PA63 (ET33_12875), A0A084H279 (GS18_0201580), A0A090ZLC1 (DJ90_5725), A0A0A1PYL7 (cypE_2 BN1110_04876), A0A0A2TAG9 (N782_10060), A0A0A2VBX0 (N780_07960), A0A0A51410 (N781_06660), A0A0B0D3P2 (LD39_06645), A0A0C2W4W6 (KR50_09340), A0A0D1LX75 (QU41_04860), A0A0D1XUB7 (AF333_17620 SAMN04487909_12329), A0A0D3V954 (UB51_10470), A0A0D5NK70 (VN24_14405), A0A0D7P697 (UP09_13525), A0A0F0HJ47 (UK12_28185), A0A0G3A308 (AA314_07761 ATI61_110119), A0A0G3URC8 (M444_30060), A0A0H2MDJ0 (VPARA_39910), A0A0J6CP59 (AB986_01510), A0A0K1E5F6 (CMC5_001820), A0A0K9GTW0 (AC625_11385), A0A0M0G514 (AF331_10625), A0A0M0KW01 (AMD01_17850), A0A0M1NV37 (AM233_19525), A0A0M2K7T9 (WN67_04050), A0A0M2VXT6 (XI25_00730), A0A0N1NK20 (OK074_8588), A0A0P0SI46 (AD017_28255), A0A0P6W4C2 (AM506_07150), A0A0Q0UDZ8 (Clow_01433), A0A0Q4RGX9 (ASF12_06290), A0A0Q6YRJ6 (ASC80_20745), A0A0Q7XLC3 (ASD54_14600), A0AOS3Q061 (cypE GJW-30_1_03917), A0A0T1WCC9 (ASD37_11505), A0A0U1NXC3 (BN000_02620), A0A0U2N4I1 (AUC31_06375), A0A0U5B146 (cypE CB4_02158), A0A0X3WPA3 (ADL30_10725), A0A101UDY9 (AQJ58_26195), A0A109D2V5 (APY03_1051), A0A109LQ32 (AUC45_10860), A0A126ZCQ6 (AX767_08380), A0A163YBJ0 (A4A58_11730), A0A168JDG6 (PBAT_23580), A0A172TQ32 (SY83_13600), A0A172ZMF6 (AR543_14735), A0A176JB29 (AS29_000975), A0A176JB54 (AS29_000980), A0A177L627 (AWH49_14945), A0A1B8UT13 (BBG47_11345), A0A1B8UVF4 (BBG47_07225), A0A1B9F1J9 (A3Q37_00061), A0A1B9Z374 (LMTR3_24710), A0A1C2DNC7 (QV13_15635), A0A1E3L8V5 (PTI45_00418), A0A1G6QNM8 (SAMN05444679_106173), A0A1G8JEE9 (SAMN05192534_13212), A0A1H0Q8Y0 (SAMN05444050_5939), A0A1H0TJ78 (SAMN04487981_1264), A0A1H0V9Z1 (SAMN04487981_13741), A0A1H1LC32 (SAMN04489717_0239), A0A1H210Y6 (SAMN05216210_3468), A0A1H3XU53 (SAMN05421743_102234), A0A1H7VPN6 (SAMN05192533_10152), A0A1H8Q8R1 (SAMN02990966_02107), A0A1H9VL77 (SAMN05518684_11165), A0A111CL73 (SAMN03159496_05870), A0A1I3V171 (SAMN04488498_101150), A0A1I4IK08 (SAMN03159341_10782), A0A1I4PEK5 (SAMN04487943_11127), A0A1I4TU36 (SAMN05216207_10034), A0A115ULZ8 (SAMN05443579_118154), A0A1161615 (SAMN05216203_1859), A0A1J6X0V3 (BHE18_03685), A0A1K2FPM7 (STEPF1_02755), A0A1M6L688 (SAMN05443507_102112), A0A1M6Q4K4 (SAMN05443637_10390), A0A1M7SY96 (SAMN05444170_0437), A0A1Q2L111 (B0X71_13980), A0A1Q9PR93 (BTR25_16705), A0A1R1F2L4 (BK138_07355), A0A1T4L7J1 (SAMN02745126_01398), A0A1U7C1L0 (cypB BSF38_00176), A0A1V2QSA7 (ALI22I_10070), A0A1W2E560 (SAMN06297251_12160), A0A1X7DEM9 (B1B01_05585 BEH 16435 CJ485_22905 SAMN06296056_1011142), A0A1X7HBC5 (SAMN05661091_2343), A0A1X7N5G7 (SAMN02982922_1222), A0A1Z2KV95 (cypD_E SMD11_0205), A0A231RDS7 (B1A99_17375), A0A239VCC0 (SAMEA4475696_00816), A0A244EDG1 (A8M77_10545 A8M77_11100), A0A244EIR3 (A8M77_01210), A0A250ICW3 (MEBOL_002509), A0A263DQR4 (CFP66_24710), A0A268SK39 (CHI14_06580), A0A269W044 (CHH75_13665), A0A2A8SE92 (CN326_08785), A0A2A8SEL8 (CN326_08120), A0A2G1XMP2 (BLA24_06890 CYQ11_27040), A0A2G9DLZ7 (CTU88_27515), A0A2P2GQD8 (VO63_11390), A0A2S0U9W6 (DCC85_10595), A0A2S5G8F8 (C4B60_17035), A0A2T4U4T2 (C6Y45_11680), A0A2T4VPV1 (DAT35_10130), A0A2U1F6T2 (C8D89_11046), A0A2U1FG79 (C8D89_104372), A0A2W1LR86 (DNH61_03115), A0A2Z2KXV1 (B9T62_31485), A0A326UAP9 (EI42_02036), A0A328VIS3 (A4R35_20075), A0A364K1P5 (DL897_15280), A0A371PKP2 (DX130_06935), A0A372J101 (D0Z06_13680), A0A3A1QVK4 (D3H55_14255), A0A3E0GXB8 (BCF44_12597), A0A3L7JUP2 (D9X91_14235), A0A3N0C1C8 (D7003_09235), A0A3N5ABX1 (EDD92_4651), A0A3R9ZS69 (EJI00_15105), A0A3S9PQ10 (EKH77_27745), A0A3S9PS68 (EKH77_32370), A0A401ZGJ9 (KDAU_33080), A0A402A324 (KTT_34090), A0A402ALY8 (KDK_38550), A0A402B865 (KDA_29860), A0A419HIJ5 (D5S17_19555), A0A429WNI3 (EJI01_15905), A0A431TEI8 (EJP69_27665), A0A438ALF8 (EKE94_01700), A0A444LMM3 (EPK99_04510), A0A4D7QFV0 (E8L99_00395), A0A4P6JTA2 (EPA93_22135), A0A4Q2LDG6 (EBB07_34750), A0A4Q4XGQ8 (DL771_010807), A0A4Q5J0G2 (ETU37_11125), A0A4Q5NSI4 (EON83_18030), A0A4R4ZV42 (E1263_02970), A0A4Y6UQ61 (FFV09_02160), A0A4Y9PEU1 (E4P39_06005), A0A4Z0H3A7 (E4663_07725), A0A505DRY4 (FGD71_000810), A0A511DIP7 (PA7_25470), A0A516RJ96 (FH965_38590), A0A519FE23 (EOP80_11240), A0A542ZFV2 (FB474_0555), A0A554VTT5 (B1A87_001145), A0A558BTC6 (FNH05_23835), A0A559J184 (FPZ44_11325), A0A559J476 (FPZ44_10985), A0A5C4VXA4 (FHP29_10275), A0A5C8PN17 (FHP25_14365), A0A5D0CY51 (FRY98_04665), A0A5J4KLX3 (KDW_30270), A0A5J6HQU3 (CP975_32150), A0A5N8X984 (FNH08_01600), A0A5R9FCR5 (FCL54_03950), A0A6A7MDF0 (GEV13_27110), A0A6G9GV75 (HA039_07265), A0A6H0CES5 (HB370_00985), A0A615WMU1 (GKC29_24830), A0A6L7HJT4 (cypD GBAA_3221), A0A6M8HLN3 (HN018_03630), A0A6P0CDJ4 (GV827_12580), A0A6P2E477 (cypE E5CHR_00447), A0A7C9RFC1 (G4V63_09410), A0A7G8W3T3 (H7F36_07395), A0A718EPC9 (ccbrp13_34750), A0A7K2FMA1 (GTW40_16935), A0A7K3GIE5 (GTW93_06065), A0A7L4XXE3 (GPZ77_02600), A0A7L4YPV3 (EK0264_12835), A0A7M2SKC6 (IM697_43900), A0A7M2SX44 (IM697_19985), A0A7R7CHA3 (MesoLjLc_31160), A0A7R7HBW7 (MesoLjLc_41300), A0A7W5B281 (FHS18_004602), A0A7W7C9J0 (HNR67_003171), A0A7W7LP01 (FHS39_002234), A0A7W7XFF4 (GGE06_007826), A0A7W8A5T1 (HNR40_005608), A0A7X2IX52 (GJU40_01125), A0A7X3K123 (EDM21_20380), A0A7Y0B813 (HHL19_20215), A0A7Y0KKY1 (HH311_17320), A0A814ISZ5 (NZN594_LOCUS35014 XKC960_LOCUS17278), A0A814PDN7 (BJG266_LOCUS17465 QVE165_LOCUS20287 QVE165_LOCUS20500), A0A814UU23 (NZN594_LOCUS60082 XKC960_LOCUS25655), A0A814WFX7 (GPM918_LOCUS23845 SRO942_LOCUS23843), A0A816EL15 (NFQ510_LOCUS26323 NZN594_LOCUS60666 XPS770_LOCUS24245), A0A818NJ81 (DUG663_LOCUS19931 NZN594_LOCUS35972 XPS770_LOCUS17577), A0A839DU69 (FHX42_001817), A0A840NP20 (BJ969_005939), A0A840W1X1 (HNR07_001199), A0A844Z6B2 (GRI35_05335), A0A848DPP3 (HF519_22475), A0A850H634 (HUO12_00010), A0A8J3HXT7 (KSX_06760), A0A8J3IKA0 (KSF_053910), A0A8J3MS10 (KSX_10230), A0A8J6MJ11 (H7249_02685), A0Y8J6 (GP2143_14381), A3WHN9 (NAP1_09657), A8FDP1 (BPUM_1680), A9AZL6 (Haur_2522), C8X7L1 (Namu_2612), C8XB87 (Namu_5109), C9YVP1 (SCAB_5931), D2B0C9 (Sros_6421), D2VNQ7 (NAEGRDRAFT_58772), D3EC66 (GYMC10_2763), D5DYE1 (BMQ_3237), D6KF56 (SSTG_05748), D6TEN2 (Krac_9955), D6U5T4 (Krac_0936), D9WB37 (SSOG_00543), E0MRC9 (R2A130_2972), E3E5G2 (PPSC2_17420), F4D1V6 (Psed_5892), F5LIY9 (HMPREF9413_3023), F7QKY7 (CSIRO_2234), H3SM19 (PDENDC454_23039), H3SPT4 (PDENDC454_27975), H8H2P3 (cypD DGo_PB0521), I0JHR8 (HBHAL_1310), 18AFG7 (A374_16363), 19C4C6 (WSK_2725), K6EB81 (BABA_04574), K6XDJ3 (KILIM_051_00160), L0DPU7 (Sinac_6636), L5N6V8 (D479_10211), 008336 (cypB cyp102A3 yrhJ BSU27160), 008394 (cypD cyp102A2 yetO yfnJ BSU07250), P14779 (cyp102A1 cyp102 BG04_163), Q2NDX9 (ELI_00100), Q65GU6 (cypE BL02398), Q81BF4 (CYP102A5 BC_3211), Q89R90 (blr2882), S9NZD8 (D187_007563), S9QX36 (D187_001021), V9GFP3 (JCM10914_3709), W4Q5P2 (JCM9140_2771), W5VZN0 (KALB_657), W7STP7 (KUTG_04542)

In some aspects, the cytochrome P450 monooxygenase has at least 60%, 65%, 70%, 75%, 80%, 85%, 90%, 91%, 92%, 93%, 94%, 95%, 96%, 97%, 98%, 99% or more amino acid sequence identity to one of the above described cytochrome P450 monooxygenases.

In some aspects, the variant cytochrome P450 monooxygenase can be in various forms, for example, such as an isolated preparation, as a substantially purified preparation, whole cells transformed with gene(s) encoding the polypeptide, and/or as cell extracts and/or lysates of such cells. The enzymes can be lyophilized, spray-dried, precipitated or be in the form of a crude paste, as further discussed below. In some aspects, any of variant cytochrome P450 monooxygenase expressed in a host cell can be recovered from the cells and or the culture medium using any one or more of the well known techniques for protein purification, including, among others, lysozyme treatment, sonication, filtration, salting-out, ultra-centrifugation, and chromatography.

Chromatographic techniques for isolation of the variant cytochrome P450 monooxygenase include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some aspects, affinity techniques may be used to isolate variant cytochrome P450 monooxygenase. In some aspects, for affinity chromatography purification, any antibody which specifically binds the cytochrome P450 monooxygenase polypeptide may be used. For the production of antibodies, various host animals, including but not limited to rabbits, mice, rats, etc., may be immunized by injection with a cytochrome P450 monooxygenase, or a fragment thereof. The cytochrome P450 monooxygenase or fragment may be attached to a suitable carrier, such as BSA, by means of a side chain functional group or linkers attached to a side chain functional group. In some aspects, the affinity purification can use a specific ligand bound by the cytochrome P450 monooxygenase.

### Polynucleotides and Expression Vectors

In another aspect, polynucleotides can encode any of the variant cytochrome P450 monooxygenase described herein. The polynucleotides may be operatively linked to one or more control sequences that control gene expression to create a recombinant polynucleotide capable of expressing the polypeptide. Expression constructs containing a heterologous polynucleotide encoding the variant cytochrome P450 monooxygenase can be introduced into appropriate host cells to express the corresponding variant cytochrome P450 monooxygenase polypeptide.

Accordingly, in some aspects, the polynucleotide encodes a variant cytochrome P450 monooxygenase having at least 60%, 65%, 70%, 75%, 80%, 85%, 90%, 91%, 92%, 93%, 94%, 95%, 96%, 97%, 98%, 99% or more sequence identity to a reference amino acid sequence selected from: SEQ ID NO: 1, SEQ ID NO: 3, SEQ ID NO: 5, SEQ ID NO: 7, SEQ ID NO: 9, SEQ ID NO: 11, or SEQ ID NO: 14.

The polynucleotides can be capable of hybridizing under highly stringent conditions to a reference polynucleotide sequence selected from SEQ ID NOs: 2, 4, 6, 8, 10, 12, or 14, or a complement thereof, and encodes a polypeptide having cytochrome P450 monooxygenase activity with one or more of the improved properties described herein.

In some aspects, the polynucleotides are codon optimized to fit the host cell in which the protein is being produced. For example, preferred codons used in bacteria are used to express the gene in bacteria; preferred codons used in yeast are used for expression in yeast; and preferred codons used in mammals are used for expression in mammalian cells. In some aspects, all codons need not be replaced to optimize the codon usage of the variant cytochrome P450 monooxygenase since the natural sequence will comprise preferred codons and because use of preferred codons may not be required for all amino acid residues.

In another aspect, the polynucleotide encoding a variant cytochrome P450 monooxygenase may be manipulated in a variety of ways to provide for expression of the polypeptide. The polynucleotides encoding the polypeptides can be provided as expression vectors where one or more control sequences are present to regulate the expression of the polynucleotides and/or polypeptides. Manipulation of the isolated polynucleotide prior to its insertion into a vector may be desirable or necessary depending on the expression vector. The techniques for modifying polynucleotides utilizing recombinant DNA methods are well known in the art. Guidance is provided in Sambrook et al., Molecular Cloning: A Laboratory Manual, 3^{rd} Ed., Cold Spring Harbor Laboratory Press (2001); and Current Protocols in Molecular Biology, Ausubel. F. ed., Greene Pub. Associates, (1998), with updates to 2006.

In some aspects, the control sequences include among others, promoters, enhancers, leader sequences, polyadenylation sequences, propeptide sequences, signal peptide sequences, and transcription terminators. Other control sequences will be apparent to the person of skill in the art.

Suitable promoters can be selected based on the host cells used. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene, the tac promoter, or the T7 promoter.

Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase.

Exemplary promoters for insect cells include, among others, those based on polyhedron, PCNA, OplE2, OplE1, Drosophila metallothionein, and Drosophila actin 5C. In some embodiments, insect cell promoters can be used with Baculoviral vectors.

Exemplary promoters for plant cells include, among others, those based on cauliflower mosaic virus (CaMV) 35S, polyubiquitin gene (PvUbi1 and PvUbi2), rice (Oryza sativa) actin 1 (OsAct1) and actin 2 (OsAct2) promoters, the maize ubiquitin 1 (ZmUbi1) promoter, and multiple rice ubiquitin (RUBQ1, RUBQ2, rubi3) promoters.

Exemplary promoters for mammalian cells include, among others, CMV IE promoter, elongation factor 1α-subunit promoter, ubiquitin C promoter, Simian Virus 40 promoter, and phosphoglycerate Kinase-1 promoter.

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used.

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region which directs the expressed polypeptide into the secretory pathway of a host cell of choice may be used in the present disclosure.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide can be converted to a mature active polypeptide by catalytic or autocatalytic cleavage of the propeptide from the propolypeptide. Where both signal peptide and propeptide regions are present at the amino terminus of a polypeptide, the propeptide region is positioned next to the amino terminus of a polypeptide and the signal peptide region is positioned next to the amino terminus of the propeptide region.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

In another aspect, the present disclosure is also directed to a recombinant expression vector comprising a polynucleotide encoding a variant cytochrome P450 monooxygenase, and one or more expression regulating regions such as a promoter and a terminator, a replication origin, etc., depending on the type of hosts into which they are to be introduced.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The expression vector can exist as a single copy in the host cell, or maintained at higher copy numbers, e.g., up to 4 for low copy number and 50 or more for high copy number.

In some embodiments, the expression vector contains one or more selectable markers, which permit selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus.

### Host Cells

In another aspect, the present disclosure provides a host cell comprising a polynucleotide encoding a variant cytochrome P450 monooxygenase of the present disclosure. Host cells can be prokaryotic or eukaryotic cells. Prokaryotic host cells include bacteria, e.g., eubacteria, such as Gram-negative or Gram-positive organisms, for example, any species of Acidovorax, Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, Vibrio, and Zymomonas, including, e.g., Bacillus amyloliquefacines, Bacillus subtilis, Brevibacterium ammoniagenes, Brevibacterium immariophilum, Clostridium acetobutylicum, Clostridium beigerinckii, Clostridium Beijerinckii, Clostridium saccharoperbutylacetonicum, Clostridium saccharobutylicum, Clostridium aurantibutyricum, Clostridium tetanomorphum, Enterobacter sakazakii, Bacillus cereus, Escherichia coli, Lactococcus lactis, Mesorhizobium loti, Pseudomonas aeruginosa, Pseudomonas fluorescens, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Serratia marcescens, Shigella dysenteriae, Shigella flexneri, Shigella sonnei, Staphylococcus aureus, Vibrio natriegens, and the like. Further examples of prokaryotic host cells include: Bacillus such as B. megaterium, B. lichenformis or B. subtilis; Pantoea, such as P. citrea; Pseudomonas, such as P. alcaligenes; Streptomyces, such as S. lividans or S. rubiginosus; Escherichia, such as E. coli; Enterobacter; Streptococcus; Archaea, such as Methanosarcina mazei; or Corynebacterium, such as C. glutamicum. The host cell can be a gram-positive bacterium such as, for example, strains of Streptomyces (e.g., S. lividans, S. coelicolor, or S. griseus) and Bacillus. The host cell can also be a gram-negative bacterium, such as, for example, E. coli and other enterics, or Pseudomonas sp. The host cell can be a soil bacteria including, for example, Bacillus (e.g., B. megaterium, B. thuringiensis, B. subtilis, B. mycoides, B. pumillus), Acidobacteriota, Verrucomicrobia, Burkholderia pseudomallei, rhizobium, Bateriodota, Streptomyces, Paenibacillus, Brevibacillus, Sporosarcina, Lysinibacillus, Psychrobacillus, Cohnella, Sporosarcina, Staphylococcus, Agrobacterium, Arthrobacter, Micromonospora, Actinomadura, Pseudomonas, Rhodococcus, Alcaligenes, Flavobacterium, Hyphomicrobium, Clostridium, Ralstonia, Collimonas, Nitrobacter, Geobacillus, Stenotrophomonas, Pseudonocardia, Deinococcus, Variovorax, Phenylobacterium, Bradyrhizobium, Saccharomonospora, Geodermatophilus, Pseudolabrys, Gemmatimonas, Frankia, Reyranella, Variibacter, Azotobacter, Nocardioides, Terriglobus, Microbispora, Microbacterium, Promicromonospora, Glycomyces, Amycolatopsis, Spirillospora, Actinopolyspora, Kitasatospora, Thermomonospora, Saccharothrix, Nocardiopsis, Microtetraspora, Pilimelia, Bryobacter, Rhizomicrobium, Firmicutes, Proteobacteria, Bacteroidetes, Actinobacteria, Nocardia, Actinoplanes, Flexibacter, Mycobacterium, Ralstonia, and Haliangium. The host cell can be a petroleum degrading bacterium including, for example, Achromobacter (e.g., Achromobacter xylosoxidans DN002), Acinetobacter (e.g., sp. RAG-1), Aeromonas (e.g., A. hydrophila), Agmenellum (e.g., quadruplicatum), Alcanivorax, Alcaligenes (e.g., A. xylosoxidans), Alkanindiges, Alteromonas, Arthrobacter, Bacillus (e.g., B. Megaterium, B. subtilis, B. licheniformis), Burkholderia, Cycloclasticus, Dietzia (e.g., Dietzia sp. DQ12-45-1b), Enterobacter, Gordonia sp., Kocuria, Marinobacter, Mycobacterium, Ochrobactrum, Oleispira, Pandoraea, Pseudomonas (e.g., P. aeruginosa, P. luorescens, P. putida), Rhodococcus (e.g., R. equi), Staphylococcus, Stenotrophomonas (e.g., S. maltophilia), Streptobacillus, Streptococcus, Thallassolituus, and Xanthomonas sp.

Other host cells include, for example, Paenibacillus sp. TCA20, Brevibacillus laterosporus LMG 15441, Brevibacillus laterosporus LMG 15441, Tetrasphaera japonica T1-X7, Paenibacillus tyrfis, Metabacillus indicus (Bacillus indicus), Paenibacillus macerans (Bacillus macerans), bacterium YEK0313, Pontibacillus yanchengensis Y32, Pontibacillus chungwhensis BH030062, Pontibacillus halophilus JSM 076056=DSM 19796, Halobacillus sp. BBL2006, Jeotgalibacillus campisalis, Bradyrhizobium elkanii, Aneurinibacillus migulanus (Bacillus migulanus), Paenibacillus sp. IHBB 10380, Paenibacillus beijingensis, Bradyrhizobium sp. LTSP885, Saccharothrix sp. ST-888, Archangium gephyra, Streptomyces sp. Mg1, Variovorax paradoxus, Alkalihalobacillus macyae, Chondromyces crocatus, Peribacillus loiseleuriae, Rossellomorea marisflavi, Priestia koreensis, Bacillus sp. FJAT-22058, Mycolicibacterium obuense, Paenibacillus sp. DMB20, Actinobacteria bacterium OK074, Pseudonocardia sp. EC080619-01, Rossellomorea vietnamensis, Corynebacterium lowii, Paenibacillus sp. Leaf72, Afipia sp. Root123D2, Rhizobium sp. Root149, Variibacter gotjawalensis, Mycobacterium sp. Root135, Neobacillus massiliamazoniensis, Planococcus rifietoensis, Aneurinibacillus soli, Streptomyces sp. NRRL S-1521, Streptomyces sp. DSM 15324, Variovorax sp. WDL1, Erythrobacter sp. YT30, Variovorax sp. PAMC 28711, Tardiphaga robiniae, Paenibacillus antarcticus, Paenibacillus swuensis, Paenibacillus bovis, Bacillus sp. SJS, Bacillus sp. SJS, Domibacillus aminovorans, Paenibacillus sp. KS1, Paenibacillus sp. KS1, Streptomyces sp. PTY08712, Bradyrhizobium sp. LMTR 3, Mesorhizobium hungaricum, Paenibacillus nuruki, Variovorax sp. CF079, Alteribacillus persepolensis, Afipia sp. GAS231, Streptomyces sp. cf386, Streptomyces sp. cf386, Actinopolymorpha singaporensis, Halopseudomonas salegens, Thalassobacillus cyri, Mesobacillus persicus, Rhodospirillales bacterium URHD0017, Salipaludibacillus aurantiacus, Rhizobium sp. NFR07, Mesorhizobium albiziae, Paenibacillus sp. 1_12, Gracilibacillus orientalis, Pseudonocardia ammonioxydans, Variovorax sp. PDC80, Marinobacter daqiaonensis, Rossellomorea aquimaris, Streptomyces sp. F-1, Alicyclobacillus montanus, Pseudonocardia thermophila, Bradyrhizobium erythrophlei, Planococcus lenghuensis, Bacillus sp. MRMR6, Paenibacillus rhizosphaerae, Enhydrobacter aerosaccus, Paludisphaera borealis, Saccharothrix sp. ALI-22-I, Fulvimarina manganoxydans, Priestia filamentosa, Paenibacillus uliginis N3/975, Mesorhizobium australicum, Streptomyces albireticuli, Cohnella sp. CIP 111063, Dermatophilus congolensis, Variovorax sp. JS1663, Variovorax sp. JS1663, Melittangium boletus DSM 14713

Pseudonocardia sp. MH-G8, Paenibacillus sp. 7516, Paenibacillus sp. 7541, Bacillus sp. AFS018417, Bacillus sp. AFS018417, Streptomyces cinnamoneus (Streptoverticillium cinnamoneum), Streptomyces sp. JV178, Streptomyces showdoensis, Paenibacillus sp. CAA11, Jeotgalibacillus proteolyticus, Alkalicoccus saliphilus, Vitiosangium sp. (strain GDMCC 1.1324), Actinomycetospora cinnamomea, Actinomycetospora cinnamomea, Paenibacillus sambharensis, Paenibacillus donghaensis, Thermosporothrix hazakensis, Thermogemmatispora tikiterensis, Thermoflavimicrobium daqui, Paenibacillus paeoniae, Geodermatophilus sp. LHW52908, Bacillus salacetis, Kutzneria buriramensis, Falsibacillus albus, Arthrobacter oryzae, Streptomyces sp. TLI_185, Variovorax sp. DXTD-1, Streptomyces luteoverticillatus (Streptoverticillium luteoverticillatus), Streptomyces luteoverticillatus (Streptoverticillium luteoverticillatus), Dictyobacter aurantiacus, Tengunoibacter tsumagoiensis, Dictyobacter kobayashii, Dictyobacter alpinus, Pseudonocardiaceae bacterium YIM PH 21723, Variovorax sp. MHTC-1, Variovorax gossypii, Mesobaculum littorinae, Neorhizobium lilium, Phreatobacter sp. NMCR1094, Ktedonosporobacter rubrisoli, Paenibacillaceae bacterium, Monosporascus sp. 5C6A, Nocardioides iriomotensis, bacterium, Kribbella antibiotica, Saccharibacillus brassicae, Blastococcus sp. CT_GayMR19, Halobacillus salinus, Streptomyces sporangiiformans, Pseudonocardia asaccharolytica DSM 44247=NBRC 16224, Streptomyces spectabilis, Variovorax sp, Oryzihumus leptocrescens, Arthrobacter sp. KBS0703, Amycolatopsis rhizosphaerae, Paenibacillus sp. N4, Paenibacillus sp. N4, Nocardioides albidus, Vineibacter terrae, Paenibacillus faecis, Dictyobacter vulcani, Streptomyces alboniger, Streptomyces spongiae, Pseudalkalibacillus caeni, Rhodospirillales bacterium, Streptomyces liangshanensis, Streptomyces sp. DSM 40868, Micromonospora sp. WMMC415, Bacillus anthracis, Lichenicola cladoniae, Sulfitobacter sediminilitoris, Variovorax sp. PBL-E5, Candidatus Afipia apatlaquensis, Variovorax sp. PAMC28562, Ktedonobacteria bacterium brp13, Streptomyces sp. SID4985, Streptomyces sp. SID5789, Streptomyces sp. QHH-9511, Epidermidibacterium keratini, Streptomyces ferrugineus, Streptomyces ferrugineus, Mesorhizobium sp. L-8-10, Mesorhizobium sp. L-8-10, Paenibacillus phyllosphaerae, Crossiella cryophile, Streptomyces olivoverticillatus, Streptomyces nymphaeiformis, Nonomuraea endophytica, Metabacillus lacus, Paenibacillus lutrae, Streptomyces sp. R302, Actinomycetospora sp. TBRC 11914, Rotaria sp. Silwood1, Adineta steineri, Rotaria sp. Silwood1, Didymodactylos carnosus, Rotaria sp. Silwood1, Rotaria sp. Silwood1, Halosaccharopolyspora lacisalsi, Saccharopolyspora gloriosae, Nocardiopsis metallicus, Pontixanthobacter aestiaquae, Pseudonocardia bannensis, Altererythrobacter lutimaris, Ktedonospora formicarum, Reticulibacter mediterranei, Ktedonospora formicarum, Oligoflexus sp, marine gamma proteobacterium HTCC2143, Erythrobacter sp. NAP1, Bacillus pumilus (strain SAFR-032), Herpetosiphon aurantiacus (strain ATCC 23779/DSM 785/114-95), Nakamurella multipartita (strain ATCC 700099/DSM 44233/CIP 104796/JCM 9543/NBRC 105858/Y-104) (Microsphaera multipartita), Nakamurella multipartita (strain ATCC 700099/DSM 44233/CIP 104796/JCM 9543/NBRC 105858/Y-104) (Microsphaera multipartita), Streptomyces scabiei (strain 87.22), Streptosporangium roseum (strain ATCC 12428/DSM 43021/JCM 3005/NI 9100), Naegleria gruberi (Amoeba), Geobacillus sp. (strain Y412MC10), Priestia megaterium (strain ATCC 12872/QMB 1551) (Bacillus megaterium), Streptomyces sp. e14, Ktedonobacter racemifer DSM 44963, Ktedonobacter racemifer DSM 44963, Streptomyces himastatinicus ATCC 53653 Ahrensia sp. R2A130, Paenibacillus polymyxa (strain SC2) (Bacillus polymyxa), Pseudonocardia dioxanivorans (strain ATCC 55486/DSM 44775/JCM 13855/CB1190), Paenibacillus sp. HGF7, Bradyrhizobiaceae bacterium SG-6C Paenibacillus dendritiformis C454, Paenibacillus dendritiformis C454, Deinococcus gobiensis (strain DSM 21396/JCM 16679/CGMCC 1.7299/1-0), Halobacillus halophilus (strain ATCC 35676/DSM 2266/JCM 20832/KCTC 3685/LMG 17431/NBRC 102448/NCIMB 2269) (Sporosarcina halophila), Fictibacillus macauensis ZFHKF-1, Novosphingobium sp. Rr 2-17, Neobacillus bataviensis LMG 21833, Kineosphaera limosa NBRC 100340, Singulisphaera acidiphila (strain ATCC BAA-1392/DSM 18658/VKM B-2454/MOB10), Halobacillus sp. BAB-2008, Bacillus subtilis (strain 168), Bacillus subtilis (strain 168), Priestia megaterium (strain ATCC 14581/DSM 32/CCUG 1817/JCM 2506/NBRC 15308/NCIMB 9376/NCTC 10342/NRRL B-14308/VKM B-512/Ford 19) (Bacillus megaterium), Erythrobacter litoralis (strain HTCC2594), Bacillus licheniformis (strain ATCC 14580/DSM 13/JCM 2505/CCUG 7422/NBRC 12200/NCIMB 9375/NCTC 10341/NRRL NRS-1264/Gibson 46), Bacillus cereus (strain ATCC 14579/DSM 31/CCUG 7414/JCM 2152/NBRC 15305/NCIMB 9373/NCTC 2599/NRRL B-3711), Bradyrhizobium diazoefficiens (strain JCM 10833/BCRC 13528/IAM 13628/NBRC 14792/USDA 110), Cystobacter fuscus DSM 2262, Cystobacter fuscus DSM 2262, Paenibacillus sp. JCM 10914, Alkalihalobacillus wakoensis JCM 9140, Kutzneria albida DSM 43870, Kutzneria sp. 744.

Eukaryotic host cells can include, for example, fungi, algal, plant, or mammalian cells. Fungal host cells include, for example, are fungi cells, including, but not limited to, fungi of the genera Aciculoconidium, Ambrosiozyma, Arthroascus, Arxiozyma, Ashbya, Aspergillus, Babjevia, Bensingtonia, Botryoascus, Botryozyma, Brettanomyces, Bullera, Bulleromyces, Candida, Chlamydomonas, Chrysosporium, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Fusarium, Galactomyces, Geotrichum, Guilliermondella, Hanseniaspora, Hansenula, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Neotyphodium, Neurospora, Ogataea, Oosporidium, Pachysolen, Penicillium, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichoderma, Trichosporiella, Trichosporon, Trigonopsis, Tsuchiyaea, Udeniomyces, Waltomyces, Wickerhamia, Wickerhamiella, Williopsis, Xanthophyllomyces, Yamadazyma, Yarrowia, Zygoascus, Zygosaccharomyces, Zygowilliopsis, and Zygozyma, among others. In some embodiments, the fungi is Candida albicans, Chrysosporium lucknowense, Fusarium graminearum, Fusarium venenatum, Hansenula polymorpha, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Schizosaccharomyces pompe, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phaffia rhodozyma), or a filamentous fungi, e.g. Trichoderma, Aspergillus sp., including Aspergillus nidulans, Aspergillus niger, Aspergillus oryzae, Aspergillus phoenicis, Aspergillus carbonarius, and the like.

The host cell can be an algae species and/or a photosynthetic, or non-photosynthetic, microorganism from Agmenellum, Amphora, Anabaena, Ankistrodesmus, Asterochloris, Asteromonas, Astephomene, Auxenochlorella, Basichlamys, Botryococcus, Botryokoryne, Boekelovia, Borodinella, Brachiomonas, Catena, Carteria, Chaetoceros, Chaetophora, Characiochloris, Characiosiphon, Chlainomonas, Chlamydomonas, Chlorella, Chlorochytrium, Chlorococcum, Chlorogonium, Chloromonas, Chrysosphaera, Closteriopsis, Cricosphaera, Cryptomonas, Cyclotella, Dictyochloropsis, Dunaliella, Ellipsoidon, Eremosphaera, Eudorina, Euglena, Fragilaria, Floydiella, Friedmania, Haematococcus, Hafniomonas, Heterochlorella, Gleocapsa, Gloeothamnion, Gonium, Halosarcinochlamys, Hymenomonas, Isochrysis, Koliella, Lepocinclis, Lobocharacium, Lobochlamys, Lobomonas, Lobosphaera, Lobosphaeropsis, Marvania, Monoraphidium, Myrmecia, Nannochloris, Nannochloropsis, Navicula, Nephrochloris, Nitschia, Nitzschia, Ochromonas, Oocystis, Oogamochlamys, Oscillatoria, Pabia, Pandorina, Parietochloris, Pascheria, Phacotus, Phagus, Phormidium, Platydorina, Platymonas, Pleodorina, Pleurochrysis, Polulichloris, Polytoma, Polytomella, Prasiola, Prasiolopsis, Prasiococcus, Prototheca, Pseudochlorella, Pseudocarteria, Pseudotrebouxia, Pteromonas, Pyrobotrys, Rosenvingiella, Scenedesmus, Schizotrichium, Spirogyra, Stephanosphaera, Tetrabaena, Tetraedron, Tetraselmis, Thraustochytrium, Trebouxia, Trochisciopsis, Ulkenia, Viridiella, Vitreochlamys, Volvox, Volvulina, Vulcanochloris, Watanabea, or Yamagishiella. The host cell can be Botryococcus braunii, Prototheca krugani, Prototheca moriformis, Prototheca portoricensis, Prototheca stagnora, Prototheca wickerhamii, Prototheca zopfii, or Schizotrichium sp. The host cell can be a fungi species from Aciculoconidium, Ambrosiozyma, Arthroascus, Arxiozyma, Ashbya, Aspergillus, Babjevia, Bensingtonia, Botryoascus, Botryozyma, Brettanomyces, Bullera, Bulleromyces, Candida, Chlamydomonas, Chrysosporium, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Fusarium, Galactomyces, Geotrichum, Guilliermondella, Hanseniaspora, Hansenula, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Neotyphodium, Neurospora, Ogataea, Oosporidium, Pachysolen, Penicillium, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichoderma, Trichosporiella, Trichosporon, Trigonopsis, Tsuchiyaea, Udeniomyces, Waltomyces, Wickerhamia, Wickerhamiella, Williopsis, Xanthophyllomyces, Yamadazyma, Yarrowia, Zygoascus, Zygosaccharomyces, Zygowilliopsis, and Zygozyma, among others. The fungi host cell can be Candida albicans, Chrysosporium lucknowense, Fusarium graminearum, Fusarium venenatum, Hansenula polymorpha, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Schizosaccharomyces pompe, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phaffia rhodozyma), or a filamentous fungi, e.g. Trichoderma, Aspergillus sp., including Aspergillus nidulans, Aspergillus niger, Aspergillus oryzae, Aspergillus phoenicis, Aspergillus carbonarius. The host cell can be a strain of the species Prototheca moriformis, Prototheca krugani, Prototheca stagnora or Prototheca zopfii and in other embodiment the cell has a 16S rRNA sequence with at least 70, 75, 80, 85, 90, 95 or 99% sequence identity (Ewing A, et al (2014). J. Phycol. 50:765-769).

Plant host cells include, for example, cells of monocotyledonous or dicotyledonous plants including, but not limited to, alfalfa, almonds, asparagus, avocado, banana, barley, bean, blackberry, brassicas, broccoli, cabbage, cannabis, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis species, woody plants such as coniferous and deciduous trees, an ornamental plant, a perennial grass, a forage crop, flowers, other vegetables, other fruits, other agricultural crops, herbs, grass, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.) The term “plants” refers to all physical parts of a plant, including seeds, seedlings, saplings, roots, tubers, stems, stalks, foliage and fruits. Plant host cells can phytoremediate petroleum, including, for example, Agropyron cristatum, Astragalus adsurgens, biochar, Caragana korshinskii, Echinacea purpurea, Epipremnum aureum, Fawn (Festuca arundinacea Schreb), Festuca ovina, Fire Phoenix (a combined F. arundinacea), Gaillardia aristata, Imperata cylindrica, Leguminous plant Acacia seiberiana Tausch, Lolium perenne, mycorrhizae, Mucuna bracteate, Medicago sativa, Pteris vittata, and Purple Nutsedge.

### Introduction of Polynucleotides to Host Cells

Polynucleotides for expression of the variant cytochrome P450 monooxygenase may be introduced into cells by various methods known in the art. Techniques include among others, electroporation, biolistic particle bombardment, liposome mediated transfection, calcium chloride transfection, microinjection, recombinant viral transfection, and protoplast fusion. The introduced nucleic acids may be integrated into chromosomal DNA or maintained as extrachromosomal replicating sequences. General transformation techniques are known in the art (see, e.g., Current Protocols in Molecular Biology, F. M. Ausubel et al. eds, Chapter 9 (1987); Sambrook et al., Molecular Cloning: A Laboratory Manual, 3rd Ed., Cold Spring Harbor Laboratory Press, N.Y. (2001); and Campbell et al., Curr Genet. 16:53-56, 1989; each publication incorporated herein by reference).

Polynucleotides for expression of the variant cytochrome P450 monooxygenase may be introduced into host cells by transfection (e.g., Gorman, et al. Proc. Natl. Acad. Sci. 79.22 (1982): 6777-6781, which is incorporated by reference in its entirety for all purposes), transduction (e.g., Cepko and Pear (2001) Current Protocols in Molecular Biology unit 9.9; DOI: 10.1002/0471142727.mb0909s36, which is incorporated by reference in its entirety for all purposes), calcium phosphate transformation (e.g., Kingston, Chen and Okayama (2001) Current Protocols in Molecular Biology Appendix 1C; DOI: 10.1002/0471142301.nsa01cs01, which is incorporated by reference in its entirety for all purposes), calcium chloride and polyethylene glycol (PEG) to introduce recombinant DNA into microalgal cells (see Kim et al., (2002) Mar. Biotechnol. 4:63-73, which reports the use of this method to transform Chlorella ellipsoidea protoplasts, and which is incorporated by reference in its entirety for all purposes), cell-penetrating peptides (e.g., Copolovici, Langel, Eriste, and Langel (2014) ACS Nano 2014 8 (3), 1972-1994; DOI: 10.1021/nn4057269, which is incorporated by reference in its entirety for all purposes), electroporation (e.g Potter (2001) Current Protocols in Molecular Biology unit 10.15; DOI: 10.1002/0471142735.im1015s03 and Kim et al (2014) Genome 1012-19. doi:10.1101/gr.171322.113, Kim et al. 2014 describe the Amaza Nucleofector, an optimized electroporation system, both of these references are incorporated by reference in their entirety for all purposes), microinjection (e.g., McNeil (2001) Current Protocols in Cell Biology unit 20.1; DOI: 10.1002/0471143030.cb2001s18, which is incorporated by reference in its entirety for all purposes), liposome or cell fusion (e.g., Hawley-Nelson and Ciccarone (2001) Current Protocols in Neuroscience Appendix 1F; DOI: 10.1002/0471142301.nsa01fs10, which is incorporated by reference in its entirety for all purposes), mechanical manipulation (e.g. Sharon et al. (2013) PNAS 2013 110 (6); DOI: 10.1073/pnas. 1218705110, which is incorporated by reference in its entirety for all purposes), biolistic methods (see, for example, Sanford, Trends in Biotech. (1988) 6:299 302, U.S. Pat. No. 4,945,050, which is incorporated by reference in its entirety for all purposes), Lithium Acetate/PEG transformation (Gietz and Woods (2006) Methods Mol. Biol. 313, 107-120) and its modifications, which is incorporated by reference in its entirety for all purposes, or other well-known techniques for delivery of nucleic acids to host cells. Once introduced, the nucleic acids can be expressed episomally, or can be integrated into the genome of the host cell using well known techniques such as recombination (e.g., Lisby and Rothstein (2015) Cold Spring Harb Perspect Biol. March 2; 7 (3). pii: a016535. doi: 10.1101/cshperspect.a016535, which is incorporated by reference in its entirety for all purposes), non-homologous integration (e.g., Deyle and Russell (2009) Curr Opin Mol Ther. 2009 August; 11 (4): 442-7, which is incorporated by reference in its entirety for all purposes) or transposition (as described above for mobile genetic elements). The efficiency of homologous and non-homologous recombination can be facilitated by genome editing technologies that introduce targeted single or double-stranded breaks (DSB). Examples of DSB-generating technologies are CRISPR/Cas9, TALEN, Zinc-Finger Nuclease, or equivalent systems (e.g., Cong et al. Science 339.6121 (2013): 819-823, Li et al. Nucl. Acids Res (2011): gkr188, Gaj et al. Trends in Biotechnology 31.7 (2013): 397-405, all of which are incorporated by reference in their entirety for all purposes), transposons such as Sleeping Beauty (e.g., Singh et al (2014) Immunol Rev. 2014 January; 257 (1): 181-90. doi: 10.1111/imr.12137, which is incorporated by reference in its entirety for all purposes), targeted recombination using, for example, FLP recombinase (e.g., O'Gorman, Fox and Wahl Science (1991) 15: 251 (4999): 1351-1355, which is incorporated by reference in its entirety for all purposes), CRE-LOX (e.g., Sauer and Henderson PNAS (1988): 85; 5166-5170), or equivalent systems, or other techniques known in the art for integrating the nucleic acids of the invention into the eukaryotic cell genome.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Techniques for transforming a wide variety of higher plant species are well known and described in the technical and scientific literature. See, e.g., Weising (1988) Ann. Rev. Genet. 22:421-477; U.S. Pat. No. 5,750,870, which are both incorporated by reference in their entirety for all purposes.

### Transfer of Nucleic Acids

Transmission of genes between bacteria can occur through transformation, transduction, or conjugation. Conjugation efficiency is most commonly quantified by the ratio of the number of transconjugants (i.e., recipient cells that have received a plasmid from a donor cell) to the number of donors or recipients at the beginning of conjugation. This is the conjugation frequency, and is also known as the conjugation rate. The conjugation frequency is affected by various biotic and abiotic factors including, for example, such as growth phase, cell density, donor-to-recipient ratio, carbon and metal concentrations, temperature, pH, and mating time.

ssDNA transfer by conjugation is ubiquitous in the bacterial and archaebacterial world and relies on a dedicated cell envelope spanning DNA transfer machinery ancestral to T4SS (type IV secretion systems) which translocate virulence determining effector proteins into target cells. Approximately 10-20 proteins (fewer in Gram positive bacteria) constitute the building blocks of the T4SS dedicated to ssDNA and protein transfer. The T4S machinery and additional proteins required for DNA transfer and replication are encoded by conjugative plasmids or integrative conjugative elements.

Plasmids carrying genes that code for all the machinery needed to form a mating pair and transfer the plasmid to the recipient are called self-transmissible plasmids, whereas plasmids that require the help of transfer machinery encoded on other plasmids in the donor bacterium to achieve this are called mobilizable plasmids. Conjugative plasmids include or can be derived from, for example, F-plasmids, other Gram Negative conjugative plasmids, broad host range plasmids RP4 and R388, pKM101, pAMB1, pAT191, pAM714, pAM771, pCAL1, pCAL2, pCF10, Tn1545, Tn916, pYD1, ROR-1, pIP72, pOX38, RIP1a, R1, R1drd19, pCVM29188_146, TP123, R64, pHUSEC41-1, pES1, TP114, pIP6, RPI/RP4, pRK24, RK2, R388, R6K, R100, R6-5, ColB2-K77.

Conjugative plasmids belong to 23 different incompatibility groups: B, C, D, E, FI, FII, FIII, FIV, H, Ia, I2, Ic, Id, If, J, K, M, N, P, T, V, W and X. Alternatively, conjugative plasmids can be grouped into compatibility groups using Inc/rep typing. In 2018, there were 28 Inc types in Enterobacteriaceae, 14 in Pseudomonas and approximately 18 in Staphylococcus. Still further, conjugative plasmids can be grouped using PCR based typing where different regions (e.g., rep genes, iterons, RNAI) are used. Conjugative plasmids within a group can stably coexist in the same host cell.

Conjugative plasmids grouped by Inc type show that several groups are promiscuous and have broad host ranges. IncA/C is a group conjugative, self-transferable plasmids with a size range of 40-230 kb. IncA/C conjugative plasmids have a broad host range which include members of Beta-, Gamma-, and Deltaproteobacteria classes. IncN (e.g., pCU1), IncP (e.g., RK2), and IncW group conjugative plasmids also have a broad host range including, for example, Beta-, Gamma-, Deltaproteobacteria, and Bacteroidetes classes. IncQ, IncU and PromA conjugative plasmids also have a broad host range including, for example, at least Alpha-, Beta-, and Gammaproteobacteria classes. In phylogeny, class is above order, which is above family, which is above genus, which is above specie. The conjugative plasmids from the broad host range Inc groups are compatible with many, many bacterial host cells. For example, IncP- and IncPromA-conjugative plasmids can transfer to a broad range of soil bacteria including recipients from eleven (11) different bacterial phyla.

Agrobacterium can be used to transfer genetic material from suitable vectors into recipient plant cells. Zhang et al, A highly efficient Agrobacterium-mediated method for transient gene expression and functional studies in multiple plant species, Plant Commun. 1:1000028 (2020) doi.org/10.1016/j.xpic.2020.1000028; Gelvin, Agrobacterium-mediated plant transformation: the biology behind the gene-jockeying tool, Microbiol. Mol. Biol. Rev. 67:16-37 (2003), each of which is incorporated by reference in its entirety for all purposes. The T-DNA region in Ti plasmids can be transferred from Agrobacterium into recipient, host plant cells. Agrobacterium can transfer DNA to a remarkably broad group of organisms including numerous dicot, monocot angiosperm species, gymnosperms, fungi, including yeasts, ascomycetes, and basidiomycetes.

Superspreader mutations, that dramatically enhanced the conjugation efficiency of conjugative plasmids belonging to diverse incompatibility groups. The first superspreader mutation was characterized in the F plasmid, which carries an IS3 insertion sequence into the finO gene. FinO inactivation destabilizes the FinP-traJ mRNA duplex, thus resulting in the upregulation of traJ and the constitutive expression of tra genes. This naturally occurring mutation accounts for the enhanced transfer efficiency of the F plasmid compared with the related IncF plasmids R100, R6-5, and R1, in which the FinOP regulatory system is still active. More recently, genetically induced superspreader mutations of several resistance plasmids have been isolated in laboratory settings. In the IncI plasmid pESBL, which is associated with extended-spectrum β-lactamase production in Enterobacteria, inactivation of the Hft locus triggered the overexpression of conjugative pili and 20-fold enhancement of the transfer efficiency. In the Citrobacter freundii IncM group plasmid pCTX-M3 that carries the blaCTX-M-3 gene, the deletion of two genes (orf35 and orf36) resulted in the enhanced expression of tra genes and increased plasmid transfer. Another example was reported in the Gram-positive broad host range (Inc18) plasmid pIP501, which is involved in the propagation of vancomycin resistance from Enterococci to methicillin-resistant strains of Staphylococcus aureus. In this case, the deletion of the traN gene encoding the small cytosolic protein TraN (unrelated to the F TraN protein) resulted in the upregulation of transfer factors and the enhancement of the transfer efficiency. Insertion of the Tn1999 transposon into the tir (transfer inhibition of RP4) gene of the IncL/M-type plasmid pOXA-48a, responsible for the dissemination of specific extended-spectrum β-lactamase genes in Enterobacteriaceae, increases the transfer efficiency by 50-100-fold without affecting traM expression levels.

Conjugation requires that a donor call meet a recipient cell, form a conjugative pilus, and attach to the surface of the recipient cell. The probability of mating-pair formation is influenced by the density of donors and recipients, their motility, and the structure of the environment (i.e., liquid versus solid, or structure of the filter). Once a mating pair has been successfully formed, a copy of the plasmid has to be transferred to the recipient, and the pilus should remain intact until this process is finished. Once inside the recipient, the plasmid should escape degradation by restriction endonucleases of the recipient which recognize restriction sites on the plasmid, and host factors should be able to ensure plasmid replication and equal distribution of the plasmid copies among the two daughter cells during cell division.

The conjugation efficiency can also be affected by plasmids that are already present in the recipient bacterium. They can stabilize mating pairs and increase the conjugation efficiency, or decrease mating-pair formation and make it more difficult for other related plasmids to enter the recipient. Plasmids in the recipient can inhibit stable maintenance of other plasmids if they use the same replication-control mechanism. Based on the different replication-control mechanisms, 28 different incompatibility (Inc) groups are recognized for plasmids in Enterobacteriaceae. (Rozwandowicz et al., J. Antimicrob. Chemother, 73:1121-37 (2018), which is incorporated by reference in its entirety for all purposes) The presence of genes coding for replication-control mechanisms correlates with the presence of genes needed for conjugation, and therefore may correlate with differences in conjugation efficiency.

Decreasing taxonomic relatedness between donor and recipient bacteria is associated with a lower conjugation frequency in liquid matings, but not in substrate matings. In a substrate mating, the donor and recipient cells are fixed in space such that conjugation is limited to neighboring cells. Under these conditions, mating-pair formation does not play an important role in limiting conjugation, because conjugation to related recipients will be efficient at first and will then saturate when all neighboring recipients have received the plasmid. Conjugation to less-related recipients might be less efficient, but because the bacteria are more fixed in space, conjugation will continue for a longer time, and cease when all neighboring recipients have received the plasmid. Competition for mating-pair formation can be less important in a substrate because the mating pairs are fixed in space. As a result, the difference in conjugation frequency to more-related versus less-related recipients will decrease over time, and at later time points relatedness does not to influence the conjugation frequency.

Substrates that can increase conjugation to less related recipient cells include, for example, biofilms, soil, the gut of an animal, and other environments were the host and recipient cells are fixed in space so that conjugation can occur between neighboring cells.

Transduction is a common tool used to stably introduce a foreign gene into a host cell's genome. Transduction vectors, methods of producing transducing particles, and methods for transducing host cells are known in the art. Broad host range, transducing phage include, for example, SN-T, STP4-a, vB_SPUM_SP116, SH6, SH7, SaFB14, KFS-SE1, fmb-p1, SS3e, ZCSE2, myPSH2311, ST32, vB_ValP_IME271, JHP, vB_PcaM_CBB.

Diverse soils including clay soil, forest soil, desert soil, or fertile mollisol have bacteria in the range of 10⁴ to 10⁹ cells per gram. Phage have been found in soil in the range of 0 to 10⁷ plaque forming unites per gram. In aquatic systems including freshwater lakes, streams, marine waters, and river mud bacterial counts were in the range of 10³ to 10⁹ bacteria per milliliter. Phage have been found in high concentrations in aquatic environments ranging from 10³ to 10⁸ plaque forming units per milliliter. Thus, transduction of genetic material is a common feature in soil and aquatic communities of bacteria.

In natural transformation, competent cells take up DNA and incorporate it into their genome. Naturally transformable bacteria develop competence and take up DNA under in situ conditions. And DNA has been shown to persist in the environment and to be available for uptake by competent cells. Many species of naturally competent bacteria have been identified in soil and aquatic sediments.

### Methods for Using the Variant Cytochrome P450 Monooxygenase

The variant cytochrome P450 monooxygenases disclosed herein can be used to insert one atom from molecular oxygen into an exceptionally broad range of substrates while reducing the other atom to water. Under hypoxic conditions variant cytochrome P450 monooxygenases can perform reductive reactions, contributing electrons to drive reductive elimination reactions. P450s can catalyze dehalogenation and denitration of a range of environmentally persistent pollutants including halogenated hydrocarbons and nitroamine explosives. The variant cytochrome P450 monooxygenases can catalyze the initial oxidation step in the catabolism of persistent organic pollutants such as polycyclic aromatic hydrocarbons (PAHs). The variant cytochrome P450 monooxygenases can hydroxylate hydrophobic, high-molecular weight PAHs such as naphthalene (C₁₀H₈), phenanthrene (C₁₄H₁₀), pyrene (C₁₆H₁₀), and chrysene (C₁₈H₁₂). Beyond oxidation of hydrocarbons, variant cytochrome P450 monooxygenases can perform oxidative dehalogenations. Variant cytochrome P450 monooxygenases can oxidize 1,2-dichloropropane to chloroacetone and can hydroxylate polychlorinated benzenes. Variant cytochrome P450 monooxygenases can also catalyze reductive halide elimination reactions when their usual co-substrate, molecular oxygen, is absent.

In an aspect, the variant cytochrome P450 monooxygenases disclosed herein can be used in the bioremediation of persistent organic pollutants. Such persistent organic pollutants include, for example, polycyclic aromatic hydrocarbons, crude oil, certain components of crude oil (e.g., dodecane), PFAs (polyfluoroalkyl compounds), PCBs (polychlorinated biphenyl compounds), pesticides, and halogen containing organic compounds.

When used in the remediation of crude oil, the variant cytochrome P450 monooxygenases disclosed herein can be combined with almA (flavin binding monooxygenase), and xylE (catechol dioxygenase). Optionally, alkB (alkane hydroxylase) and ndo (naphthalene dioxygenase) can also be used. A suitable host cell can be engineered with nucleic acids encoding a variant cytochrome P450 monooxygenase, a flavin binding monooxygenase (almA) and a catechol dioxygenase (xylE). The expression of these three nucleic acids in the host cell allow that host cell to use crude oil, and/or dodecane as a carbon source. These engineered host cells can be used to directly remediate the crude oil, and/or the engineered host cell can be used to transfer the nucleic acids encoding the variant cytochrome P450 monooxygenase, the flavin binding monooxygenase (almA), and the catechol dioxygenase (xylE) to indigenous organisms (e.g., soil bacteria or plants).

Two approaches to remediating crude oil using indigenous bacterial populations with catabolic genes of interest are (1) through controlled mating (in vitro) and re-release or (2) direct application of engineered host cells with the catabolic genes of interest an in situ transfer of the catabolic genes. This approach can be applied to water/marine samples (substituting water for soil).

In another aspect, the variant cytochrome P450 monooxygenase described herein can be used in a process or method for remediating PFAs. Generally, the method comprises contacting or incubating PFAs, with a variant cytochrome P450 monooxygenase in combination with a dehalogenase such as, for example, flouroacetate dehalogenase (fac dex), haloacetate dehalogenase, tetrachloroethylene reductive delahogenase (pceA), haloalkane dehalogenase (dhaA). The reaction can occur in vitro, for example, in cell free systems, or in vivo, for example where the process uses a host cell, such as a microbial organism, expressing a variant P450 monooxygenase.

In another aspect, the variant cytochrome P450 monooxygenase described herein can be used in a process or method for remediating PCBs. Generally, the method comprises contacting or incubating PCBs, with a variant cytochrome P450 monooxygenase in combination with laccases, peroxidases, haloalkane dehalogenases, and/or hydrolytic dehalogenases. The reaction can occur in vitro, for example, in cell free systems, or in vivo, for example where the process uses a host cell, such as a microbial organism, expressing a variant P450 monooxygenase.

In an aspect, the variant cytochrome P450 monooxygenase described herein can be attached or associated with a substrate (e.g., a filter) for treating fluids (e.g., waste water). In this aspect the variant cytochrome P450 monooxygenase may be attached to the substrate with a spacer or tether of desired length. Such tethers or spacers are well known in the art and can, for example, be 1-10 carbons in length (e.g., ethyl, propyl, butyl, etc.).

In carrying out the variant cytochrome P450 monooxygenase mediated methods described herein, the engineered polypeptide may be added to the reaction mixture in the form of a purified enzyme, whole cells transformed with gene(s) encoding the enzyme, and/or as cell extracts and/or lysates of such cells. Host cells transformed with gene(s) encoding a variant cytochrome P450 monooxygenase or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, and the like). Any of the cell preparations may be stabilized by crosslinking using known crosslinking agents, such as, for example, glutaraldehyde or immobilization to a solid phase.

The gene(s) encoding the variant cytochrome P450 monooxygenase can be transformed into host cell separately or together into the same host cell. For example, one set of host cells can be transformed with gene(s) encoding one variant cytochrome P450 monooxygenase and another set can be transformed with gene(s) encoding another variant cytochrome P450 monooxygenase. Both sets of transformed cells can be utilized together in the reaction mixture in the form of whole cells, or in the form of lysates or extracts derived therefrom. A host cell can be transformed with gene(s) encoding multiple variant cytochrome P450 monooxygenase. The engineered polypeptides can be expressed in the form of secreted polypeptides and the culture medium containing the secreted polypeptides can be used for the reaction.

Various ranges of suitable reaction conditions can be used in the methods, including but are not limited to, substrate loading, pH, temperature, buffer, solvent system, polypeptide loading, and reaction time. Further suitable reaction conditions for reacting substrate compound to product compound using an variant cytochrome P450 monooxygenase can be readily optimized in view of the guidance provided herein and by routine experimentation that includes, but is not limited to, contacting the variant cytochrome P450 monooxygenase and substrate compound under experimental reaction conditions of concentration, pH, temperature, and solvent conditions, and detecting the product compound. Substrate compound in the reaction mixtures can be varied, taking into consideration, for example, the desired amount of product compound, the effect of substrate concentration on enzyme activity, stability of enzyme under reaction conditions, and the percent conversion of substrate to product.

Suitable assays to detect products from the variant cytochrome P450 monooxygenase can be performed using well known methods. Product synthesis can be analyzed by methods such as GC-MS (Gas Chromatography-Mass Spectroscopy) and LC-MS (Liquid Chromatography-Mass Spectroscopy) or other suitable analytical methods using routine procedures well known in the art. A typical assay method is described in, for example, Manual on Hydrocarbon Analysis (ASTM Manula Series), A. W. Drews, ed., 6th Ed., American Society for Testing and Materials, Baltimore, Maryland, 1998.

Where whole cells can used in a method for remediating crude oil, PFAs, PCBs, halogenated compounds, or components of crude oil, samples from cultures grown for each engineered strain to be tested can be examined for degradation of the target molecule as well as various intermediates in the degradation pathway.

B. megaterium compositions can be made combining a B. megaterium comprising a nucleic acid encoding a mutant P450 (e.g., SEQ ID NO: 2, 4, 6, 8, 10, and/or 12) with other microorganisms such as, for example, Anaerolineae, Bacteroidales, Bacteroides, Bacteroides sp900766195, Bacteroidetes, Chlamydomonas, Dehalococcoides, Dehalococcoides mccartyi, Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, Rhodopseudomonas palutus. B. megaterium compositions can also be made with B. megaterium that does not contain a mutant P450, combined with other microorganisms listed above. B. megaterium compositions can be made combining a B. megaterium comprising a nucleic acid encoding a mutant P450 (e.g., SEQ ID NO: 2, 4, 6, 8, 10, and/or 12) with two or more of the preceding microorganisms.

B. megaterium compositions can be made combining a B. megaterium and/or a B. megaterium comprising a nucleic acid encoding a mutant P450 (e.g., SEQ ID NO: 2, 4, 6, 8, 10, and/or 12) with a microorganism that can provide one or more of the following: biosurfactant production, salt tolerance, plant growth promoting, spore forming, anaerobic, aerobic, photosynthetic, alkane degradation, PAH degradation, alkene degradation, pH tolerance, polymer production, and/or nitrogen fixation. Exemplary microorganisms that can provide one of more of the above include, for example, Anaerolineae, Bacteroidetes, Dehalococcoides, Pseudomonas, and/or Rhodococcus. Exemplary microorganisms also include those described above as microorganisms to combine with B. megaterium comprising a nucleic acid encoding a mutant P450.

These B. megaterium compositions can also include buffers (e.g., TRIS, ammonium nitrate, potassium phosphate, sodium bicarbonate, HEPES, RIPA, MOPS, MES hydrate, etc.), salts, (e.g., NaCl, potassium sulfate, silver nitrate, ammonium nitrate, sodium sulfate, etc.), metals (e.g., copper, cobalt, iron, lead, nickel, silver, zinc, etc.), chelators (e.g., EDTA, EDTA: Fe(III), hydroxyethylethylenediaminetriacetic (HEDTA), ethylenediamine, EDDHA, Dimethylglyoxime, iminodiacetic acid, trisodium dicarboxymethyl alaninate, DOTA, crown ethers, etc.), other additives, for example, chitin, biological polymers, other polymers, rhamnolipids, other lipids, biotin, streptavidin, yeast extract, tryptone, other growth media, cryoprotectants, etc.

Exemplary biological polymers can include, for example, polysaccharides, polypeptides, hyaluronan, chitin, chitosan, heparin, chondroitin sulfate, keratin sulfate, dermatan sulfate, dextran, xanthan, levan, glycogen, gellan, glucuronan, succinoglycan, alginate, carrageenan, fucoidan, ulvan, glycosaminoglycans, polyamides (PA), polyesters (PE), poly(ester-amide) s (PEA) s, polyurethanes (PU) s, poly(depsipeptide) s (PDP) s, collagen, fibrin, fibrinogen, gelatin, silk, elastin, myosin, keratin, actin, etc.

Other polymers can include, for example, Poly(α-hydroxy esters) including PCL (polycaprolactone), PGA (polyglycolic acid), PLA (poly lactic acid), and their copolymer PLGA (polylactic-co-glycolic acid) and poly(ethers) including PEO (polyethylene oxide) and PEG (polyethylene glycol), PVA (polyvinyl alcohol), and PU (polyurethane). Still other polymers can include, for example, PDLLA (poly-d-lactic acid), PLLA (poly-l-lactic acid), PPF (polypropylene fumarate), PBS (polybutylene succinate), PHB (polyhydroxy butyrate), etc.

Other lipids can include, for example, phospholipids, triglycerides, saturated lipids, polyunsaturated lipids, omega-3 polyunsaturated fatty acids, omega-6 polyunsaturated fatty acids, steroids, sterols, cholesterol, monoglycerides, diglycerides, glycerolipids, glycerophospholipids, sphingolipids, saccharolipids, polyketides, 1,2-dilinolenoyl-sn-glycero-3-phosphocholine (DLPC), 1,2-dilinoleoyl-sn-glycero-3-phosphocholine (DLinPC), 1,2-dioleoyl-sn-glycero-3-phosphocholine (DOPC), phosphatidylcholine (PC), phosphatidylethanolamine (PE), phosphatidic acid (PA), and phosphatidylinositol (Pl), sphingomyelin (SM).

Other growth media can include, for example, Luria broth (LB), M9 minimal media, Bacillus Medium from HIMEDIA Laboratories, Inc., other commercially available growth medias for Bacillus, and media disclosed in Cho et al., Optimization of culture media for Bacillus species by statistical experimental design methods, 2009, Korean J. Chemical Engineering vol 26, pp. 754-759, which is hereby incorporated by reference in its entirety for all purposes.

Exemplary cryoprotectants can include, for example, Sucrose, Trehalose, Mannitol, Sorbitol, Glucose, Maltose, Polyethylene glycol (PEG), Dimethyl sulfoxide (DMSO), Glycerol, Polyvinylpyrrolidone (PVP), Hydroxyethyl starch (HES), Polyvinyl alcohol (PVA), Dextran, Polyethyleneimine (PEI), Hydroxypropyl cellulose (HPC), Hydroxypropyl methylcellulose (HPMC), Albumin (e.g., bovine serum albumin), Gelatin, Casein, Xylitol, Erythritol, Polyvinylpyrrolidone-vinyl acetate copolymer (PVPVA), PEGylated compounds, Betaine, Hydroxypropyl-beta-cyclodextrin (HPBCD), Inulin, Raffinose, Polyethylene oxide (PEO), Methylcellulose, Dextransucrase, Inositol, Antifreeze proteins (AFP), Lactose, Propylene glycol, Trimethylglycine (betaine), Hydroxyethyl cellulose (HEC), Polydextrose, Cellulose, Polyacrylic acid, Sodium alginate, Polyaspartic acid, Chitosan, Polyethyleneimine (PEI), Polyhydroxybutyrate (PHB), Polyhydroxyalkanoates (PHA), Ethylene glycol, Pectin, Carrageenan, Sodium carboxymethylcellulose (CMC), and/or Mannose.

B. Megaterium compositions can include, for example, B. megaterium and/or B. megaterium comprising a nucleic acid encoding a mutant P450 (e.g., SEQ ID NO: 2, 4, 6, 8, 10, and/or 12), and optionally one or more other microorganism, and optionally a buffer, and/or salt, and/or metal, and/or chelator, and/or growth media, and/or cryoprotectant. B. Megaterium compositions can be B. megaterium comprising a nucleic acid encoding a mutant P450 (e.g., SEQ ID NO: 2, 4, 6, 8, 10, and/or 12), Anaerolineae, and one or more of Bacteroidales, Bacteroides, Bacteroides sp900766195, Bacteroidetes, Chlamydomonas, Dehalococcoides, Dehalococcoides mccartyi, Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Bacteroidales, and one or more of Bacteroides, Bacteroides sp900766195, Bacteroidetes, Chlamydomonas, Dehalococcoides, Dehalococcoides mccartyi, Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Bacteroides sp900766195, and one or more of Bacteroidetes, Chlamydomonas, Dehalococcoides, Dehalococcoides mccartyi, Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Chlamydomonas, and one or more of Dehalococcoides, Dehalococcoides mccartyi, Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Dehalococcoides, and one or more of Dehalococcoides mccartyi, Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Dehalococcoides mccartyi, and one or more of Dehalococcoidia, Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Dehalococcoidia, and one or more of Pseudomonas, Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas, and one or more of Pseudomonas farris, Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas farris, and one or more of Pseudomonas fluorescens, Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas fluorescens, and one of Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas fluorescens, and one or more of Pseudomonas frederiksbergensis, Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas frederiksbergensis, and one or more of Pseudomonas guariconensis, Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas guariconensis, and one or more of Pseudomonas khazarica, Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. Megaterium compositions can be B. megaterium and/or B. megaterium comprising a nucleic acid encoding a mutant P450 (e.g., SEQ ID NO: 2, 4, 6, 8, 10, and/or 12), Pseudomonas khazarica, and one or more of Pseudomonas mosselii, Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Pseudomonas mosselii, and one or more of Pseudomonas parafulva, Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Pseudomonas parafulva, and one or more of Pseudomonas putida, Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Pseudomonas putida, and one or more of Pseudomonas simiae, Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Pseudomonas simiae, and one or more of Pseudomonas sp000765155, Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Pseudomonas sp000765155, and one or more of Pseudomonas veronii, Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Pseudomonas veronii, and one or more of Rhodococcus, Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus, and one or more of Rhodococcus sp. 77-32, Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. 77-32, and one or more of Rhodococcus sp. 065240, Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. 065240, and one or more of Rhodococcus sp. KY1, Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. KY1, and one or more of Rhodococcus sp. M4, Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. M4, and one or more of Rhodococcus sp. MB 5655, Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. MB 5655, and one or more of Rhodococcus sp. MA7205, Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. MA7205, and one or more of Rhodococcus sp. Q1, Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. Q1, and one or more of Rhodococcus sp. YH3-3, Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus sp. YH3-3, and one or more of Rhodococcus erythropolis, Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus erythropolis, and one or more of Rhodococcus equi, Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus equi, and one or more of Rhodococcus globerulus, Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus globerulus, and one or more of Rhodococcus hoagie, Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus hoagie, and one or more of Rhodococcus josti, Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus josti, and one or more of Rhodococcus kyotonensis, Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus kyotonensis, and one or more of Rhodococcus opacus, Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus opacus, and one or more of Rhodococcus pseudokoreensis, Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus pseudokoreensis, and one or more of Rhodococcus pyridinivorans, Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus pyridinivorans, and one or more of Rhodococcus qingshengii, Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus qingshengii, and one or more of Rhodococcus rhodochrous, Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus rhodochrous, and one or more of Rhodococcus ruber, Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus ruber, and one or more of Rhodococcus wratislavlensis, Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus wratislavlensis, and one or more of Rhodococcus zopfii, Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus zopfii, and one or more of Rhodococcus Zopf 1891, Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), Rhodococcus Zopf 1891, and one or more of Rhodopseudomonas acidophilus, Rhodopseudomonas capsulatus, Rhodopseudomonas palustris, and Rhodopseudomonas palutus.

B. megaterium compositions can be B. megaterium and/or B. megaterium comprising a nucleic acid encoding a mutant P450 (e.g., SEQ ID NO: 2, 4, 6, 8, 10, and/or 12), and one of Rhodopseudomonas acidophilus, Rhodopseudomonas capsulatus, Rhodopseudomonas palustris, and Rhodopseudomonas palutus.

The B. megaterium compositions can be freeze dried, lyophilized, or frozen for longer term storage or shelf life. Working stocks of the B. megaterium compositions can be liquid media, slants, and plates stored at a variety of temperatures for immediate use. The B. megaterium compositions can be formulated or processed for ease of administration, storage and application, e.g., frozen, lyophilized, suspended (suspension formulation) or powdered; and processed for application as a spray (e.g., hand sprayer, backpack sprayer, truck sprayer or plane spraying), through an irrigation system (e.g., in irrigation water), or injected into the ground as a liquid, time release formulation or device, or in a solid. The B. megaterium compositions can be formulated in a matrix. Such matrices can lead to time delayed release of the B. megaterium composition into the environment.

Application of the B. megaterium composition to the surface of an area to be treated can allow the B. megaterium composition to penetrate to a depth of from 30-50 feet, 30-40 feet, 20-50 feet, 20-40 feet, 20-30 feet, 10-30 feet, or 10-20 feet. Application of the B. megaterium composition to the surface of an area to be treated can allow the B. megaterium composition to penetrate to a depth of at least 10 feet, 20 feet, 30 feet, 40 feet, or 50 feet. Application of the B. megaterium composition to the surface of an area to be treated can allow the B. megaterium composition to penetrate to a depth of at most 10 feet, 20 feet, 30 feet, 40 feet, or 50 feet. Application of the B. megaterium composition to the surface of an area to be treated can allow the B. megaterium composition to penetrate to the depth of the water-table. Application of the B. megaterium composition to the surface of an area to be treated can allow the B. megaterium composition to penetrate into the water-table.

When the B. megaterium composition is injected or applied into the ground the composition can spread from 10-20 feet from the site of application (e.g., the bore hole). Injection of the B. megaterium composition can treat the area around the injection site for at least 10 feet, 20 feet, 30 feet, 40 feet or 50 feet. Injection of the B. megaterium composition can treat the area around the injection site for at most 10 feet, 20 feet, 30 feet, 40 feet or 50 feet.

B. megaterium compositions can be used in the bioremediation of persistent organic pollutants. Such persistent organic pollutants include, for example, polycyclic aromatic hydrocarbons, crude oil, certain components of crude oil (e.g., dodecane), PFAs (polyfluoroalkyl compounds), PCBs (polychlorinated biphenyl compounds), pesticides, and halogen containing organic compounds.

B. megaterium compositions can be used in a process or method for remediating PFAs. Generally, the method comprises engineering an organism for the B. megaterium compositions, with a dehalogenase such as, for example, flouroacetate dehalogenase (fac dex), haloacetate dehalogenase, tetrachloroethylene reductive delahogenase (pceA), haloalkane dehalogenase (dhaA). Alternatively, the B. megaterium composition can include an organism that is capable of degrading PFAS. The resulting B. megaterium composition can be applied to the water or soil to be remediated of PFAS.

Various features and embodiments of the disclosure are illustrated in the following representative examples, which are intended to be illustrative, and not limiting. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: Synthesis and Screening of Variant Cytochrome P450 Monooxygenase

48 selected amino acid residues were mutagenized and assembled into single-site variant libraries (SSVLs) where 19 variants were made for each position corresponding to the 19 other possible amino acids for that site. This was repeated for all 48 positions generating a total of 912 variants which were then subjected to screening via growth assays and GC/MS followed by Sanger sequencing to identify mutations in variants. Out of the 48 residues selected for mutagenesis 17 were chosen due to being previously reported in the literature (R47, Y51, M354, L437, F87, A264, F42, T268, P382, S383, A384, I385, P386, Q387, W96, F393, L188). 17 were selected for being within 5 angstroms of the substrate bound to the active site (L20, P25, V26, L29, A74, L75, V78, F181, T260, I263, A328, P329, A330, T438) and the remaining 17 residues were chosen semi-randomly for their proximity to other functional residues that could play an auxiliary role in binding or catalysis (V48, T49, A82, L86, V178, A184, R255, F261, L353, I401, F405), in addition to several other distant residues selected for their potential to impact enzyme flexibility and its conformational changes (H138, H236, M237, E252, A290, A295).

E. coli harboring plasmids with variant enzymes were cultured for 2 days and diluted by a factor of 10 in minimal media containing 1 or 2% of the target substrate (crude oil, dodecane or Benzopyrene (BaP) respectively). Growth assays with the target substrate as the sole carbon source were run for 5 days taking OD600 (cell density) readings approximately every 24 hours. Samples with higher growth rates compared to the wildtype enzyme were sequenced and collected for downstream analysis of degradation products via GC/MS. The growth results are shown in Table 2:

TABLE 2
Growth with Variant Cytochrome P450 Monooxygenase

| | Dodecane % Improved | Crude Oil % Improved |
|---|---|---|
| Variant | Compared to WT | Compared to WT |
| F42W | 79.43% | 46.89% |
| H138A | 56.72% | −4.37% |
| L86N | 49.27% | 12.81% |
| T49S | 74.06% | −82.97% |
| V78W | 16.39% | 67.77% |
| L75N | 8.43% | 65.45% |
| T49A | 8.15% | ND |
| F42T | 9.46% | ND |
| F42V | 13.32% | ND |
| F42C | 1.78% | ND |
| F42G | 16.16% | ND |
| F42I | 8.14% | ND |
| F42R | 3.08% | ND |
| F42D | 52.12% | ND |

ND = no data

All of the cytochrome P450 monooxygenase variants improved growth on dodecane, and four (4) of the variants improved growth on crude oil.

Bacillus megaterium with the P450 variants F42W, T49S, L75N, V78W, L86N and H138A were obtained, and these B. megaterium were used to degrade crude oil. The degradation of crude oil by these variants in B. megaterium are shown in Table 3 below:

TABLE 3
Degradation of Oil by P450 Variants

| Variant | % Degraded over 10 days | Oil Degradation (ml) |
|---|---|---|
| Wildtype BM3 | 22.40% | 0.448 |
| F42W | 32.15% | 0.643 |
| T49S | 29.02% | 0.5804 |
| L75N | 38.70% | 0.774 |
| V78W | 28.04% | 0.5608 |
| L86N | 34.20% | 0.684 |
| H138A | 34.71% | 0.6942 |

### Example 2: Expression of Variant Cytochrome P450 Monooxygenase

The variant Cytochrome P450 monooxygenase polypeptides were introduced into Bacillus, Pseudomonas, E. coli, Paraburkholderia, and Rhodopseudomonas using several approaches, including, for example, plate-based mating methods; conjugative strains of E. coli; and protoplast transformation.

### Example 3: Activity Assay for Variant Cytochrome P450 Monooxygenase

Cytochrome P450 monooxygenase activity was determined as follows. E. coli or B. megaterium cells transformed with variant enzyme are grown in minimal media with crude oil (2% to 20%) as the carbon source. The cultures are monitored for growth by OD measurements, and after 3-5 days the supernatant is tested by Mass Spec.

When 5% crude oil is used with minimal media, the growth of E. coli or B. megaterium with the various variant cytochrome P450 monooxygenases is measured, and the supernatants were tested by Mass Spec for components. These results show that the variants from Example 1 grow on the minimal media with crude oil, and reduce the size of components in the crude oil.

### Example 4: Bioremediation of Crude Oil

Nucleic acids encoding variant cytochrome P450 monooxygenase of Example 1 are engineered into an expression vector with almA (flavin binding monooxygenase), xylE (catechol dioxygenase). The expression vector is engineered to so that the vector can be transferred by a conjugation system. The nucleic acid is transferred to an E. coli host cell that is engineered to express the components of the conjugation system. The host cells with the expression vector are expanded to make about 40-45 g of freeze-dried bacteria per 100 m² of surface are for soil to be treated.

The expanded E. coli cells with the vector are applied to soil that has been contaminated with crude oil. The E. coli cells transfer the expression vector to soil bacteria in the contaminated soil, and these organisms grow using the components of the crude oil as a carbon source and/or degrading other components of the crude oil.

### Example 5: Degradation of Crude Oil

Petroleum assays were conducted using M9 minimal media excluding any carbon source. Cell cultures grown overnight were back diluted 10-fold and made up 10% of the total volume. Crude oil was added to reach a concentration of 1%-5% v/v. Assays were conducted for 5 days with OD600 measured daily.

In a controlled, 5 day experiment, roughly 36% of total petroleum hydrocarbons in liquid media were broken down by the B. megaterium composition. Table 3 below shows a GC-MS analysis of subsets of petroleum products in samples after 5 days.

TABLE 4
GC-MS Analysis of Petroleum Degradation

| Peak | Ret | Avg Area | Avg Area | % | | | |
|---|---|---|---|---|---|---|---|
| # | Time | Control | Helios | Decrease | | | Carbon |
| 1 | 3.013 | 9912 | 5921 | 40.3 | Toluene | BTEX | 7 |
| 2 | 8.923 | 7783 | 2064 | 73.5 | Nonane | Alkane | 9 |
| 4 | 13.247 | 20111 | 13659 | 32.1 | Decane | Alkane | 10 |
| 12 | 16.910 | 35129 | 25060 | 28.7 | Undecane | Alkane | 11 |
| 20 | 20.105 | 43360 | 32620 | 24.8 | Dodecane | Alkane | 12 |
| 28 | 22.952 | 75248 | 62015 | 17.6 | Tridecane | Alkane | 13 |
| 37 | 25.624 | 78024 | 59514 | 23.7 | Tetradecane | Alkane | 14 |
| 42 | 28.138 | 53918 | 42321 | 21.5 | Pentadecane | Alkane | 15 |
| 46 | 30.492 | 40893 | 29806 | 27.1 | Hexadecane | Alkane | 16 |
| 48 | 31.587 | 12165 | 9809 | 19.4 | Heptadecane | Alkane | 17 |
| 59 | 36.188 | 35554 | 30361 | 14.6 | Eicosane | Alkane | 20 |
| 61 | 36.657 | 22442 | 14490 | 35.4 | Heneicosane | Alkane | 21 |
| 63 | 37.045 | 26012 | 16568 | 36.3 | Docosane | Alkane | 22 |
| 65 | 37.389 | 13684 | 8364 | 38.9 | Tricosane | Alkane | 23 |
| 66 | 37.690 | 18141 | 10521 | 42 | Tetracosane | Alkane | 24 |
| 69 | 38.250 | 9055 | 5287 | 41.6 | Hexacosane | Alkane | 26 |
| 70 | 38.508 | 4926 | 3916 | 29.5 | Heptacosane | Alkane | 27 |
| 71 | 38.766 | 4416 | 3054 | 29.2 | Octacosane | Alkane | 28 |

### Example 6: Bioremediation of Crude Oil in Pots

4″ pots were filled with 65 g of soil and artificially contaminated with 5% crude oil. Pots were seeded with three germinated barley seeds to mimic vegetation cover. Pots were treated with either water (“control”) or the B. megaterium composition. At the end of 30 days, the soil in each pot was homogenized and ˜50 g from each pot was analyzed.

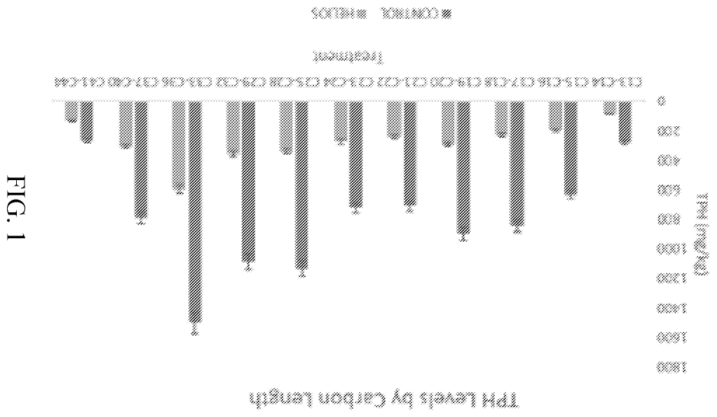

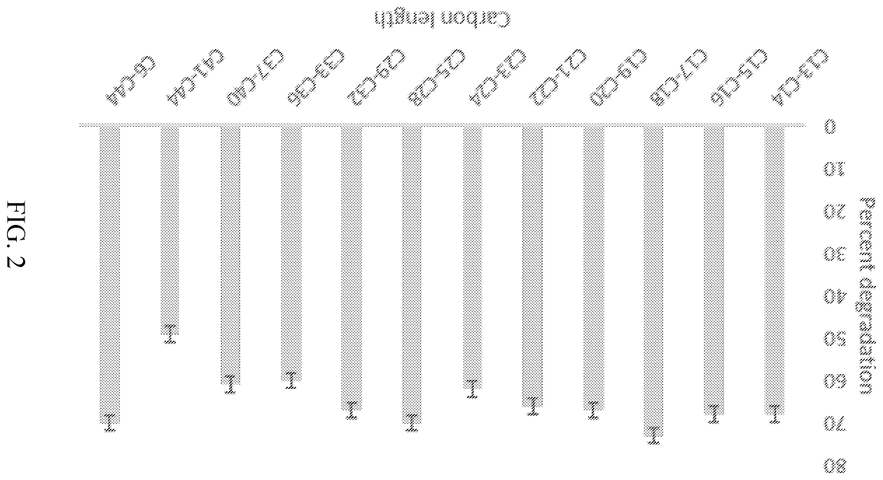

The B. megaterium composition degraded about 70% of the petroleum hydrocarbons in the contaminated soil. FIG. 1 shows the amount of petroleum by carbon length present before and after treatment with the B. megaterium composition. FIG. 2 shows the removal of crude oil in percent amounts by carbon length.

### Example 7: Bioremediation of Crude Oil in the Field

A field-trial was conducted in Fairbanks, AK. Two 1 m² plots were designated with stakes and rope (“control” and “treated”). The treated plot was sprayed with the B. megaterium composition once using a conventional hand-held sprayer. Soil samples were collected before the study began and after 30 days to measure petroleum hydrocarbon levels and to analyze microbial community composition.

Soil samples were taken before and after 30 days of treatment. The B. megaterium composition was still present in the soil at 30 days, and 51% of total petroleum hydrocarbons (870 mg/kg to 430 mg/kg) were removed after 30 days by the B. megaterium composition.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the scope of the invention(s) of the disclosure.

What is claimed is:

1. A variant cytochrome P450 monooxygenase comprising an improved degradation of a crude oil as compared to a wild-type cytochrome Pe450 monooxygenase.

2. The variant cytochrome P450 monooxygenase of claim 1, wherein an amino acid is changed at one or more positions selected from the group consisting of a L20, a P25, a V26, a L29, a F42, a R47, a V48, a T49, a Y51, an A74, a L75, a V78, an A82, a L86, a F87, a W96, a H138, a V178, a F181, an A184, a L188, a H236, a M237, an E252, a R255, a F261, an A264, a T260, an I263, a T268, an A290, an A295, an A328, a P329, an A330, a L353, a M354, a P382, a S383, an A384, an 1385, a P386, a Q387, a F393, an I401, a F405, a L437, and a T438.

3. The variant cytochrome P450 monooxygenase of claim 2, wherein the variant cytochrome P450 monooxygenase has one or more changes at the following amino acid positions a F42, a T49, a L75, a V78, a L86, or a H138.

4. The variant cytochrome P450 monooxygenase of claim 2, wherein the variant cytochrome P450 monooxygenase has one or more of the following amino acid substitutions a F42W, a H138A, a L86N, a V78W, a L75N, or a T49S.

5. The variant cytochrome P450 monooxygenase of claim 1, wherein the variant cytochrome P450 monooxygenase has an amino acid sequence selected from the group consisting of SEQ ID NO: 1, 3, 5, 7, 9, or 11.

6. The variant cytochrome P450 monooxygenase of claim 1, wherein a nucleic acid encoding the variant cytochrome P450 monooxygenase is codon optimized.

7. The variant cytochrome P450 monooxygenase of claim 1, wherein the variant cytochrome P450 monooxygenase is in a soil bacteria.

8. The variant cytochrome P450 monooxygenase of claim 1, wherein the variant cytochrome P450 monooxygenase is in a Bacillus megaterium.

9. A polynucleotide encoding the variant cytochrome P450 monooxygenase of claim 1.

10. The polynucleotide of claim 9, wherein the polynucleotide encodes a variant cytochrome P450 monooxygenase having an amino acid substitution at one or more positions selected from the group consisting of a L20, a P25, a V26, a L29, a F42, a R47, a V48, a T49, a Y51, an A74, a L75, a V78, an A82, a L86, a F87, a W96, a H138, a V178, a F181, an A184, a L188, a H236, a M237, an E252, a R255, a F261, an A264, a T260, an I263, a T268, an A290, an A295, an A328, a P329, an A330, a L353, a M354, a P382, a S383, an A384, an 1385, a P386, a Q387, a F393, an 1401, a F405, a L437, and a T438.

11. The polynucleotide of claim 9, wherein the polynucleotide encodes a variant cytochrome P450 monooxygenase having an amino acid substitution at one or more positions selected from the group consisting of a F42, a T49, a L75, a V78, a L86, or a H138.

12. The polynucleotide of claim 9, wherein the polynucleotide encodes a variant cytochrome P450 monooxygenase having one or more of the following amino acid substitutions a F42W, a H138A, a L86N, a V78W, a L75N, or a T49S.

13. The polynucleotide of claim 9, wherein the polynucleotide encodes a polypeptide selected from the group consisting of SEQ ID NO: 1, 3, 5, 7, 9, and 11.

14. The polynucleotide of claim 9, comprising a nucleotide sequence selected from the group consisting of SEQ ID NO: 2, 4, 6, 8, 10, and 12.

15. A vector comprising the polynucleotide of claim 9.

16. The vector of claim 15, comprising a promoter.

17. A host cell comprising a polynucleotide of claim 11.

18. The host cell of claim 17, wherein the host cell is a soil bacteria.

19. The host cell of claim 17, wherein the host cell is a Bacillus megaterium.

20. A method for remediating a crude oil, comprising the steps of: applying a cell to a soil contaminated with the crude oil, wherein the cell is a bacterial cell comprising a polynucleotide encoding the variant cytochrome P450 monooxygenase of claim 1 operably linked to a first promoter; and breaking down a component of the crude oil in the bacterial cell with the polynucleotide.
